(19) United States
(12) Patent Application Publication
(10) Pub. No.: US 2021/0379094 A1
(43) Pub. Date: Dec. 9, 2021

(54) METHOD FOR REDUCING DRUG-INDUCED NEPHROTOXICITY

(71) Applicant: Yissum Research Development Company of the Hebrew University of Jerusalem Ltd., Jerusalem (IL)

(72) Inventors: Yaakov NAHMIAS, Mevaseret Zion (IL); Avner EHRLICH, Jerusalem (IL); Aaron COHEN, Jerusalem (IL)

(73) Assignee: Yissum Research Development Company of the Hebrew University of Jerusalem Ltd., Jerusalem (IL)

(21) Appl. No.: 17/408,431

(22) Filed: Aug. 22, 2021

Related U.S. Application Data
(63) Continuation of application No. PCT/IL2020/050173, filed on Feb. 16, 2020.
(60) Provisional application No. 62/808,615, filed on Feb. 21, 2019.

(51) Int. Cl. A61K 31/7048; A61K 31/12; A61K 31/7034; A61P 13/12

(52) U.S. Cl. CPC A61K 31/7048; A61P 13/12; A61K 31/7034; A61K 31/12

(57) ABSTRACT

A method for reducing renal tissue toxicity in a subject caused by a kidney damaging agent is disclosed. The method comprises administering to the subject:
(i) a kidney damaging agent; and
(ii) an inhibitor of glucose reabsorption.

### RELATED APPLICATIONS

This application is a Continuation of PCT Patent Application No. PCT/IL2020/050173 having International filing date of Feb. 16, 2020, which claims the benefit of priority under 35 USC § 119(e) of U.S. Provisional Patent Application No. 62/808,615 filed on Feb. 21, 2019. The contents of the above applications are all incorporated by reference as if fully set forth herein in their entirety.

### FIELD AND BACKGROUND OF THE INVENTION

The present invention, in some embodiments thereof, relates to an ex vivo method of analyzing the toxic effects of agents on the kidney. The present invention further relates to methods of reducing drug-induced nephrotoxicity.

Drug-induced nephrotoxicity is an extremely common condition and is responsible for a variety of pathological effects on the kidneys. It is defined as renal disease or dysfunction that arises as a direct or indirect result of exposure to drugs. The incidence of drug-induced nephrotoxicity has been increasing with the increasing use of prescription drugs and their easy availability as over-the-counter medications especially non-steroidal anti-inflammatory drugs (NSAIDs) or antibiotics. Drug-induced acute renal failure accounts for 20% of all acute renal failure cases. Among older adults, the incidence of drug-induced nephrotoxicity may be as high as 66%, due to a higher incidence of diabetes and cardiovascular diseases compelling them to take multiple medications. Although renal impairment is often reversible, it may still require multiple interventions and hospitalization. Most of the drugs which are found to be nephrotoxic exert toxic effects by one or more common pathogenic mechanisms. These include altered intraglomerular hemodynamics, tubular cell toxicity, inflammation, crystal nephropathy, rhabdomyolysis, and thrombotic microangiopathy. Knowledge of offending drugs and their particular pathogenic mechanisms of renal injury is critical for recognizing and preventing drug-induced renal impairment.

Cyclosporine A (CsA) is a very important immunosuppressive drug: it has been widely used in transplantation and greatly improves the survival rates of patients and grafts after solid-organ transplantation. However, the chronic use of CsA associates with high incidences of nephrotoxicity and the eventual development of chronic renal failure. Indeed, nephrotoxicity is the most frequent and clinically important complication of CsA use, especially in renal-transplant patients. CsA directly affects renal tubular epithelial cells; specifically, it promotes epithelial-mesenchymal transition, inhibits DNA synthesis and induces apoptosis. The CsA-induced apoptosis correlates with the oxidative stress, endoplasmic reticulum stress and autophagy that CsA causes. In humans and animals, the liver and intestines are the main sites where CsA is metabolized. The limit of intestinal metabolism causes the poor oral bioavailability of CsA in humans. CsA metabolites are generally less cytotoxic than the parent drug. However, higher concentrations of some CsA metabolites associate with nephrotoxicity in organ-transplant patients. Notably, compared to the liver, there is much less biotransformation of CsA in the kidney. This may explain why this drug is so nephrotoxic in vivo.

Cisplatin, is a well-known chemotherapeutic drug. It has been used for treatment of numerous human cancers including bladder, head and neck, lung, ovarian, and testicular cancers. It is effective against various types of cancers, including carcinomas, germ cell tumors, lymphomas, and sarcomas. Its mode of action has been linked to its ability to crosslink with the purine bases on the DNA, interfering with DNA repair mechanisms, causing DNA damage, and subsequently inducing apoptosis in cancer cells. Dose-related and cumulative renal insufficiency, including acute renal failure, is the major dose-limiting toxicity of Cisplatin. Renal toxicity has been noted in 28% to 36% of patients treated with a single dose of 50 mg/m². It is first noted during the second week after a dose and is manifested by elevations in blood urea nitrogen (BUN) and creatinine, serum uric acid and/or a decrease in creatinine clearance. Renal toxicity becomes more prolonged and severe with repeated courses of the drug. Renal function must return to normal before another dose of Cisplatin can be given. Impairment of renal function has been associated with renal tubular damage. The administration of Cisplatin using a 6- to 8-hour infusion with intravenous hydration, and mannitol has been used to reduce nephrotoxicity. However, renal toxicity still can occur after utilization of these procedures.

Aminoglycoside antibiotics are widely used in the treatment of a variety of infections produced by Gram-negative bacteria and bacterial endocarditis. Their cationic structure, seems to have an important role in their toxicity, mostly affecting renal (nephrotoxicity) and hearing (ototoxicity) tissues in which they accumulate. In spite of their undesirable toxic effects, aminoglycoside antibiotics still constitute the only effective therapeutic alternative against germs insensitive to other antibiotics. This is primarily because of their chemical stability, fast bactericidal effect, synergy with betalactamic antibiotics, little resistance, and low cost. In spite of being one of the most nephrotoxic aminoglycoside antibiotic, gentamicin is still frequently used as a first- and second-choice drug in a vast variety of clinical situations. Moreover, gentamicin has been widely used as a model to study the nephrotoxicity of this family of drugs, both in experimental animals and human beings.

Organ-on-a-chip applications allow the fabrication of minimal functional units of a single organ or multiple organs. Relevant to the field of nephrology, renal tubular cells have been integrated with microfluidic devices for making kidneys-on-a-chip. Although still early in development, kidneys-on-a-chip have shown potential to replace traditional animal and human studies. They either focus on the filtration unit (the glomerula) or the tubular unit responsible for reabsorption and secretion. In the first type of kidney on a chip, human iPS-derived podocytes are combined with glomerular microvascular endothelial cells to make mature glomerular organoids (Hale, L. J., et al. Nat Commun 9, 5167 (2018) doi:10.1038/s41467-018-07594-z). Others developed organoids with multiple renal cell types from the glomerular and the tubular compartments but at the nephron progenitor cell level (Takasato, M., Nature 526, 564-568 (2015) doi:10.1038/nature15695; Jian Hui Low, Cell Stem Cell, Volume 25, Issue 3, 2019, Pages 373-387.e9, ISSN 1934-5909). The second type of kidney on a chip replicate the tubular system on a chip made of two chambers separated by a porous silicon membrane. The first compartment holds the proximal tubule cells, the second the endothelium (Kyung-Jin Jang, Integrative Biology, Volume 5, Issue 9, September 2013, Pages 1119-1129). In both systems, drug-induced nephrotoxicity can be studied at the end of the induction.

WO2013158143 teaches that SGLT-2 inhibitors can be used to reduce renal toxicity of glucose conjugated chemotherapeutic drugs such as glucosfamide.

Additional background art includes Lhotak et al., Am J Physiol Renal Physiol 303: F266-F278, 2012.

### SUMMARY OF THE INVENTION

According to an aspect of the present invention there is provided a method for reducing renal tissue toxicity in a subject caused by a kidney damaging agent, the method comprising administering to the subject:

(i) a kidney damaging agent; and

(ii) an inhibitor of glucose reabsorption, with the proviso that when the kidney damaging agent is glucosfamide, with the proviso that when the kidney damaging agent is glucosfamide, said inhibitor is not a sodium-glucose transport protein 2 (SGLT2) inhibitor.

According to another aspect of the present invention there is provided a method for reducing renal tissue toxicity in a subject caused by a kidney damaging agent, the method comprising administering to the subject:

(i) a kidney damaging agent; and

(ii) an agent that causes a decrease in lipid accumulation in renal tissue of the subject, thereby reducing renal toxicity in the subject, with the proviso that when the kidney damaging agent is glucosfamide, the agent that causes a decrease in lipid accumulation is not an SGLT2 inhibitor.

According to another aspect of the present invention there is provided a method for reducing renal tissue toxicity in a subject caused by a kidney damaging agent, the method comprising administering to the subject an agent that causes a decrease in lipid accumulation in renal tissue of the subject, thereby reducing renal toxicity caused by a kidney damaging agent in the subject, with the proviso that when the kidney damaging agent is glucosfamide, the agent that causes a decrease in lipid accumulation is not an SGLT2 inhibitor.

According to another aspect of the present invention there is provided a method for reducing renal tissue toxicity in a subject caused by a kidney damaging agent, the method comprising administering to the subject an inhibitor of glucose reabsorption, thereby reducing renal toxicity caused by a kidney damaging agent in the subject, with the proviso that when the kidney damaging agent is glucosfamide, the inhibitor is not an SGLT2 inhibitor.

According to another aspect of the present invention there is provided a composition comprising:

(i) a kidney-damaging agent; and

(ii) an agent that causes a decrease in lipid accumulation in renal tissue.

According to another aspect of the present invention there is provided a composition comprising:

(i) a kidney-damaging agent; and

(ii) an inhibitor of glucose reabsorption.

According to another aspect of the present invention there is provided a pharmaceutical composition comprising a pharmaceutically acceptable carrier and, as active agents:

(i) a kidney-damaging therapeutic agent; and

(ii) an agent that causes a decrease in lipid accumulation in renal tissue.

According to another aspect of the present invention there is provided a pharmaceutical composition comprising a pharmaceutically acceptable carrier and, as active agents:

(i) a kidney-damaging therapeutic agent; and

(ii) an inhibitor of glucose reabsorption.

According to embodiments of the present invention the pharmaceutical composition is for use in treating a disease for which the kidney damaging therapeutic agent is therapeutic.

According to embodiments of the present invention the kit is for use in treating a disease for which the kidney damaging therapeutic agent is therapeutic.

According to another aspect of the present invention there is provided a kit comprising:

(i) a kidney-damaging agent;

(ii) and an agent that causes a decrease in lipid accumulation in renal tissue.

According to another aspect of the present invention there is provided a kit comprising:

(i) a kidney-damaging agent;

(ii) an inhibitor of glucose reabsorption.

According to another aspect of the present invention there is provided an isolated organoid comprising mature, polarized kidney epithelial cells and endothelial cells, and wherein the organoid comprises three-dimensional longitudinal tubules having at least two openings, each organoid having at least one central lumen, wherein less than 50% of the cells of the organoid express a fetal marker.

According to another aspect of the present invention there is provided a microfluidic biosensor array comprising a plurality of wells comprising the organoids described herein.

According to another aspect of the present invention there is provided a method for determining the toxic effect of a candidate agent on the kidney, the method comprising:

(i) providing the organoid described herein;

(ii) culturing the organoid under physiological conditions in the presence of the candidate agent; and

(iii) performing real-time measurements of oxygen consumption of the organoid, wherein a decrease in oxygen consumption of the organoid in the presence of the candidate agent as compared to the oxygen consumption of the organoid in the absence of the candidate agent is indicative that the candidate agent has a toxic effect on the kidney.

According to embodiments of the invention, the subject has cancer and the kidney damaging agent is a therapeutic agent used to treat the cancer.

According to embodiments of the invention, the subject has undergone an organ or tissue transplant and the kidney damaging agent is an immunosuppressive agent.

According to embodiments of the invention, the subject has an infection and the kidney damaging agent is used to treat the infection.

According to embodiments of the invention, the kidney damaging agent is a therapeutic agent.

According to embodiments of the invention, the kidney damaging agent is a diagnostic agent.

According to embodiments of the invention, the subject does not have a metabolic disease.

According to embodiments of the invention, the subject does not have diabetes.

According to embodiments of the invention, the agent that causes a decrease in lipid accumulation in renal tissue is selected from the group consisting of an inhibitor of glucose reabsorption, a blocker of lipid synthesis and an up-regulator of lipid oxidation.

According to embodiments of the invention, the protective agent is an inhibitor of glucose reabsorption.

According to embodiments of the invention, the inhibitor of glucose reabsorption is selected from the group consisting of an inhibitor of Sodium-Glucose cotransporter 1 (SGLT1), an inhibitor of a sodium-glucose cotransporters 2 (SGLT2) and an inhibitor of GLUT2.

According to additional embodiments, the inhibitor of glucose reabsorption is selected from the group consisting of an inhibitor of Sodium-Glucose cotransporter 1 (SGLT1), and an inhibitor of GLUT2.

Thus, in one embodiment, the inhibitor is an inhibitor of a sodium-glucose cotransporters 2 (SGLT2).

According to embodiments of the invention, the inhibitor of glucose reabsorption is selected from the group consisting of Phloretin, Phlorizin and empagliflozin.

According to embodiments of the invention, the kidney damaging agent is selected from the group consisting of an NSAID, an ACE Inhibitor, an angiotensin II Receptor Blocker, an aminoglycoside antibiotic, a radiocontrast dye, cyclosporine A (CsA) and a chemotherapeutic agent.

According to embodiments of the invention, the kidney damaging agent is selected from the group consisting of cisplatin, gentamicin and Cyclosporine A.

According to embodiments of the invention, the tubules have a diameter of about 10 to 200 microns.

According to embodiments of the invention, the tubules have a length of about 100-1000 microns.

According to embodiments of the invention, the kidney cells are selected from the group consisting of: Human Kidney-2 cells (HK-2), primary Renal Proximal Tubule Epithelial Cells (RPTEC) and a combination of the two types of cells.

According to embodiments of the invention, the isolated organoid is vascularized.

According to embodiments of the invention, the isolated organoid is embedded with at least one microsensor for oxygen monitoring.

According to embodiments of the invention, the microsensor comprises an oxygen sensing nanoparticle or microparticle.

According to embodiments of the invention, the oxygen sensing nanoparticle or microparticle is loaded with a ruthenium-based dye.

According to embodiments of the invention, at least a portion of the wells comprise a microwell insert which protects cells from the negative effect of shear force.

According to embodiments of the invention, the biosensor has a three-electrode design in which the counter and reference electrodes are separate, the reference electrode is used to measure the working electrode potential without passing current through it, and the counter electrode closes the circuit.

According to embodiments of the invention, the agent in step (ii) is present at a concentration that causes less than 20% of the cells in the organoid to die in 24 hours.

According to embodiments of the invention, the agent in step (ii) is present at a concentration that causes less than 10% of the cells in the organoid to die in 24 hours.

According to embodiments of the invention, the method further comprises measuring the concentration at least one metabolite selected from the group consisting of glucose, lactate and glutamine.

According to embodiments of the invention, the method further comprises measuring the concentration at least two metabolites selected from the group consisting of glucose, lactate and glutamine.

According to embodiments of the invention, the method further comprises measuring the concentration at each of the metabolites glucose, lactate and glutamine.

According to embodiments of the invention, the method further comprises performing a metabolic flux analysis.

According to embodiments of the invention, the culturing is effected in a perfused bioreactor which is connected to a biosensor array.

According to embodiments of the invention, the biosensor array is fluidically linked to electrochemical sensors thereby directly measure metabolites in central carbon metabolism which are produced by the organoid.

According to embodiments of the invention, the candidate agent is selected from the group consisting of a therapeutic agent, a diagnostic agent, an imaging agent, a food, a cosmetic, and an agent suspected of having an environmental nephrotoxic effect.

According to embodiments of the invention, the measurements of oxygen consumption are selected from the group consisting of:

a. determination of the percentage of the reduction of the oxygen consumption caused by the agent;

b. determination of the duration of exposure to the candidate agent that causes a reduction in the oxygen consumption; and

c. determination of the concentration of the candidate agent that causes a reduction of the oxygen consumption.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E. Proximal tubule HK2 cell line models drug-induced nephrotoxicity in-vitro. (A) Phase images of renal proximal tubule epithelial cells (RPTEC) and Human Kidney-2 (HK-2) cells in a 2D monolayer and in a 3D cysts configuration. (B) Gene expression analysis in HK2 monolayers (HK2 2D) and HK2 organoids (HK2 3D) shows similar expression levels of transporters with RPTEC monolayers (RPTEC 2D) involved in renal physiology. (C) Dose-dependent toxicity curves of HK-2 cells treated with cyclosporine A, cisplatin or gentamicin for 24 hours in standard 2D cell culture. TC₅₀ values were 51.3 μM, 95.1 μM and 182.4 mM respectively. (D) Confocal microscopy images of HK2 cells, showing immunofluorescence staining of acute injury marker KIM-1 spreading from perinuclear aggregates to cytosolic and membranar expression after 24 h of sub-toxic drug exposure in standard 2D cultures. HSP60 staining suggests a disruption of the mitochondrial network at sub-toxic dose exposure. Scale bar=25 μm. (E) Quantification of KIM-1 and HSP60 expression after 24 h of nephrotoxic drugs exposure.

FIGS. 2A-F. Sub-toxic drug exposure on proximal tubule cysts induced rapid loss of functional polarity. (A) Immunofluorescence staining of sodium-potassium pump (red), a basolateral membrane marker and lectin tetragonobulus lectin (green), proximal tubule cells and apical membrane specific, counterstained with Hoechst for DNA (blue), positive in HK-2 and RPTEC monolayers. (B) Immunofluorescence staining of HK2 compared to RPTEC three-dimensional polarized cysts positive for Aquaporin-1 (green) and F-Actin (red), counterstained with Hoechst for DNA (blue). Cysts form with polarized epithelial cells, their apical membrane facing the lumen. White bar=cross section for expression profiles (C) Expression profiles for AQP1 (green), F-Actin (red) and DNA (blue) in HK-2 and RPTEC cysts. (D) Immunofluorescence staining for the acute injury marker KIM-1 in HK2 cysts after 24 h of sub-toxic drug exposure. Early toxicity can be observed in this model at very low concentrations. (E) Functional polarity assay on HK-2 cysts. Calcein AM (green) is uptaken by the cyst from the media and expelled by MDR1 (P-gp) localized on the apical membrane of the cells. After 24 h exposure of sub-toxic doses of three known nephrotoxic drugs, calcein AM is retained in the cells suggesting a drug-induced mislocalization of MDR1 at very low concentrations. (F) Quantification of the loss of functional polarity experiment. Calcein AM fluorescence is decreasing while KIM-1 expression is increasing, both in a dose dependent manner.

FIGS. 3A-H. Design of a microphysiological flux balance platform. (A) Metabolic pathways of glucose utilization in human proximal tubule cells. Flux balance analysis permits the calculation of intracellular fluxes using extracellular oxygen, glucose, lactate, glutamine and glutamate measurements. Dotted arrows note experimentally-limited fluxes. (B) 3D design of CNC-fabricated 6-unit bioreactor plate. Laser-cut disposable microwell chips containing 9 organoids are seeded with microsensors in an open configuration and then perfused until metabolic stabilization achieved. Immunofluorescent staining shows a human kidney organoid composed of LTL (green) and Na/K ATPase (red)—positive HK2 proximal tubule cells (blue). Oxygen sensors (orange) are embedded inside the microtissue (blue) during seeding. Scale bar=100 μm (C) Platform schematics. Bioreactor is loaded with tissue-embedded oxygen sensors and mounted on an Olympus IX83. OPAL-controlled modulation LED signal excites the embedded oxygen sensors. Phase shift is measured through a hardware-filtered photomultiplier (PMT). Bioreactor outflow is connected to a microfluidic biosensor array containing electrochemical sensors for glucose, lactate, glutamine and glutamate continuously adjusted according to non-specific oxidation events and changes in the ambient temperature. Sensors are connected to an on-chip potentiostat (PSTAT). All measurements (optical and electronic) are processed in real-time by single microprocessor, synchronizing the signal continuously. (D) (E) Low volume microfluidic amperometric, 8-electrode, biosensor array. Anodic oxidation of H₂O₂ on platinum produces a current rapidly (t₉₀<25 sec), while embedded catalase activity prevents cross-contamination. A 450-mV potential between the working and counter electrodes is monitored against a reference electrode to minimize background noise caused by reversible electrolysis events. (F) Photo of microfluidic biosensor array with total internal volume of 0.3-1 μL and integrated temperature sensors and PSTAT. (G) Raw measurements of glucose, lactate, glutamine, glutamate and temperature sensors of calibration measurements for different analyte concentrations. Measurements were carried automatically out under continuous flow of 2 μL/min. Air gap between samples ensure a sharp change in chemical gradient on the sensor during in calibration. (H) Amperometric calibration curves of glucose, lactate, glutamine and glutamate concentrations in bioreactor outflow. (I) Intracellular metabolic fluxes for polarized HK-2 organoid under steady state conditions. Glucose utilization in each pathway is shown as nmol/min/10⁶ cells as well as calculated ATP production (methods). Relative glucose utilization is shown as pie chart.

FIGS. 4A-C. Vascularized kidney organoids real-time monitoring of oxygen upon drug exposure in microphysiological platform. (A) Display of the unique three-dimensional structure of kidney organoids with longitudinal tubules surrounded by capillary-like structures. 3D reconstruction from multiple phase confocal images showing tubule-like elements in the organoid. HK-2 and RPTEC organoids show similar structures, immunofluorescence stainings are positives for AQP1 (green), F-Actin (red), and Na/K ATPase (green), Villin (purple) in HK2 and RPTEC organoids respectively. (B) Representative oxygen uptake over time response of HK-2 organoids exposed to increasing concentrations of Cyclosporine A, Cisplatin and Gentamicin. Dotted line notes exposure onset. (C) Time to onset (TTO) of response of HK-2 organoids to Cyclosporine A, Cisplatin and Gentamicin. All three drugs showed a dose-dependent decrease in TTO ranging from 13-22 hours in Cisplatin to 0.2-14 hours for Cyclosporine A and Gentamicin suggesting slowly accumulative lipotoxicity for standard therapeutics concentrations.

FIGS. 5A-E. Cyclosporine A induces a shift from glycolysis to lipogenesis at sub-toxic concentrations. (A) Curves of oxygen, glucose, lactate and glutamine fluxes during continuous perfusion with 12.5 μM Cyclosporine A. Oxygen uptake (black) drops by 2% only after 33 hours of continuous exposure. In contrast, while glucose uptake (red) stays constant, lactate production (green) drops dramatically after 20 h exposure. (B) Changes in lactate over glucose ratio following exposure to Cyclosporine A (blue line). Ratio drops by 30% after 20 h exposure, suggesting Cyclosporine A lately shifted glycolysis upon exposure. (C) Intracellular metabolic fluxes calculated following 0, 16, and 31 hours exposure to sub-toxic concentration Cylosporine A (>95% viability). Glucose utilization in each pathway is shown as nmol/min/10⁶ cells as well as calculated ATP production (methods). Lipogenesis increases by 37% while glycolysis and ATP production drop by 37% and 12%, respectively. (D) Relative glucose utilization is shown as a pie chart. Lipogenesis utilizes an increasing percentage of available glucose during Cyclosporine A exposure. (E) Schematics depicting the metabolic response of proximal tubule cells to Cyclosporine A. Dotted arrows note experimentally-limited fluxes, red and green arrows note up- and down-regulated fluxes, respectively. Cyclosporine A exposure shift glucose from lactate to citrate production, increasing lipogenesis after the first 20 hours of exposure.

FIGS. 6A-E. Cisplatin induces a shift from glycolysis to lipogenesis at sub-toxic concentrations. (A) Curves of oxygen, glucose, lactate and glutamine fluxes during continuous perfusion with 32 μM Cisplatin. Oxygen uptake (black) drops by 36% only after 33 hours of continuous exposure. In contrast, while glucose uptake (red) stays constant, lactate production (green) drops dramatically after 25 h exposure. (B) Changes in lactate over glucose ratio following exposure to Cisplatin (blue line). Ratio drops by 55% after 25 h exposure, suggesting Cisplatin accumulation lately shifted glycolysis upon exposure. (C) Intracellular metabolic fluxes calculated following 0, 16, and 31 hours exposure to sub-toxic concentration Cisplatin (>95% viability). Glucose utilization in each pathway is shown as nmol/min/10⁶ cells as well as calculated ATP production (methods). Lipogenesis increases by 57% while glycolysis and ATP production drop by 98% and 53%, respectively. (D) Relative glucose utilization is shown as a pie chart. Lipogenesis utilizes an increasing percentage of available glucose during Cisplatin exposure. (E) Schematics depicting the metabolic response of proximal tubule cells to Cisplatin. Dotted arrows note experimentally-limited fluxes, red and green arrows note up- and down-regulated fluxes, respectively. Cisplatin exposure shifts glucose from lactate to citrate production, increasing lipogenesis after the first 25 hours of exposure.

FIGS. 7A-E. Gentamicin induces a rapid shift from glycolysis to lipogenesis at sub-toxic concentrations. (A) Curves of oxygen, glucose, lactate and glutamine fluxes during continuous perfusion with 32 mM Gentamicin. Oxygen uptake (black) drops by 10% only after 33 hours of continuous exposure. In contrast, while glucose uptake (red) dramatically increases, lactate production (green) drops upon exposure. (B) Changes in lactate over glucose ratio following exposure to Gentamicin (blue line). Ratio drops by 85% after 10 h exposure, suggesting Gentamicin shifted glycolysis upon exposure. (C) Intracellular metabolic fluxes calculated following 0, 16, and 31 hours exposure to sub-toxic concentration Gentamicin (>95% viability). Glucose utilization in each pathway is shown as nmol/min/10⁶ cells as well as calculated ATP production (methods). Lipogenesis increases by 438% while glycolysis and ATP production drop by 65% and 29%, respectively. (D) Relative glucose utilization is shown as a pie chart. Lipogenesis utilizes an increasing percentage of available glucose during Gentamicin exposure. (E) Schematics depicting the metabolic response of proximal tubule cells to Gentamicin. Dotted arrows note experimentally-limited fluxes, red and green arrows note up- and down-regulated fluxes, respectively. Gentamicin exposure shifts glucose from lactate to citrate production, increasing lipogenesis upon exposure.

FIGS. 8A-E. Drug-induced loss of functional polarity causes glucose to accumulate in proximal tubule cells, leading to lipotoxicity in renal tissue. (A) Glucose uptake assay in HK-2 proximal tubule cells after sub-toxic Cyclosporine A, Cisplatin and gentamicin 15 h exposure. 2-NDBG (green) is a glucose analog internalized only by GLUT-2. 2-NDBG is retained and accumulates in the cells suggesting that the loss of functional polarity disrupts the shuttling of the glucose transporter. (B) Relative 2-NDBG accumulation quantification analysis shows a 2-fold increase in glucose content in HK2 cells after sub-toxic exposure of all three drugs. (C) Relative gene expression analysis from HK-2 monolayers after 48 h drug exposure. β-oxydation (UCP2, CPT2) and lipogenesis (FASN, SREBP1c, HMGCR) genes are upregulated after exposure, suggesting the excess glucose is shuttled to lipogenesis causing renal lipotoxicity. β-oxydation is increased as a coping mechanism. (D) Fluorescence micrographs and total quantification of lipid accumulation and phospholipidosis in HK2 and RPTEC monolayers after 48 h of Cyclosporine A, Cisplatin and Gentamicin. All three drugs induce significant neutral lipid (green) accumulation while Cyclosporine A induces significant phospholipidosis (red) in both primary cells and the cell line. (E) Relative neutral lipid (green) and phospholipid (red) content analysis after 48 h drug exposure

FIGS. 9A-B. Rescue experiment on HK-2 with glucose transport inhibitors, bar graphs show quantification for viable fractions (A) and lipid accumulations (B). Error bars indicate ±S.E.

FIGS. 10A-O. Human clinical study show that combining treatments with a SGLT2 inhibitor decrease nephrotoxicity outcomes. (A) Schematic of the physiological glucose transport system in proximal tubule cells. (B) Table describing the SGLT2 inhibitor used in the study, a FDA-approved Gliflozin used to lower glucose levels in the blood of type II diabetes patients. (C) 3D hPTC treated with CsA or Cisplatin in combination with the SGLT2 inhibitor (SGLT2i) show significant reduction of neutral lipids (green) and phospholipids (red) compared to treatment with CsA or Cisplatin alone. (D) Quantification of the LIVE/DEAD assay in HK2 cells show significantly less cell death in cells treated with CsA or Cisplatin in combination the SGLT2i or the Cocktail which is SGLT2i combined with Phloretin, an inhibitor of GLUT2 transport. (E) Quantification of lowest exposure levels (LEL) in kidney organoids. This concentration gives a safety margin of the drug, the lowest the LEL, the less safe is the drug over an infinite amount of time. SGLT2i raises the LEL by 2-fold for Cisplatin and by 3 orders of magnitude for CsA. (F) Histological slices of renal biopsies from patients treated with CsA or cisplatin, showing abnormal vacuolization and fatty vesicles in the tubules. (G) Table summarizing the number of patients in each group according to their treatment. Box plots showing serum creatinine levels (H)(J), Uric Acid levels (I)(K), serum calcium levels (L)(N) and lactate dehydrogenase levels (M)(O) in patients treated with CsA (blue) compared to patients treated with CsA and the SGLT2i simultaneously (red), or patients treated with Cisplatin (green) compared to patients treated with Cisplatin and the SGLT2i simultaneously (yellow). The light blue background in each graph stands for the normal physiological range of each marker in male and female patients. ***: p-value <0.001.

### DESCRIPTION OF SPECIFIC EMBODIMENTS OF THE INVENTION

The present invention, in some embodiments thereof, relates to an ex vivo method of analyzing the toxic effects of agents on the kidney. The present invention further relates to methods of reducing drug-induced nephrotoxicity.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The kidney is an essential organ tasked with glucose and fluid homeostasis, as well as the excretion of drug metabolites. Drug-induced nephrotoxicity accounts for 20% of kidney failure in the general population, with incidence of drug-induced nephrotoxicity increasing to 66% in elderly patients taking prescription medication. The proximal tubule is particularly sensitive to drug toxicity due to its role in the concentration and reabsorption of metabolites. Importantly, clinically relevant drug-induced nephrotoxicity often occurs at plasma concentrations of the drug that are below the threshold of cellular damage in vitro. Thus, the mechanism of damage is unclear.

The present inventors have now developed a novel microphysiological kidney-on-chip platform which comprises structurally and functionally mature proximal tubular organoids. The organoids show multiple longitudinal polarized tubules with evidence of apical brush borders. These three-dimensional organoids, made from Human Kidney-2 cells (HK-2), primary Renal Proximal Tubule Epithelial Cells (RPTEC) or Upcytea Proximal Tubule Cells (UPC PTC) are vascularized and may be embedded with microsensors for real-time oxygen measurement in the tissue. The organoids are then placed in a multi-well perfused bioreactor. These bioreactors are microfluidically linked to electrochemical sensors allowing for continuous measurements of the main players of central carbon metabolism (including glucose, lactate, glutamine and glutamate) from the outflow. This level of information contributes to the accuracy of metabolic flux analysis of the organoids, providing a window into the dynamic changes of their metabolism whilst in the presence of potentially nephrotoxic agents.

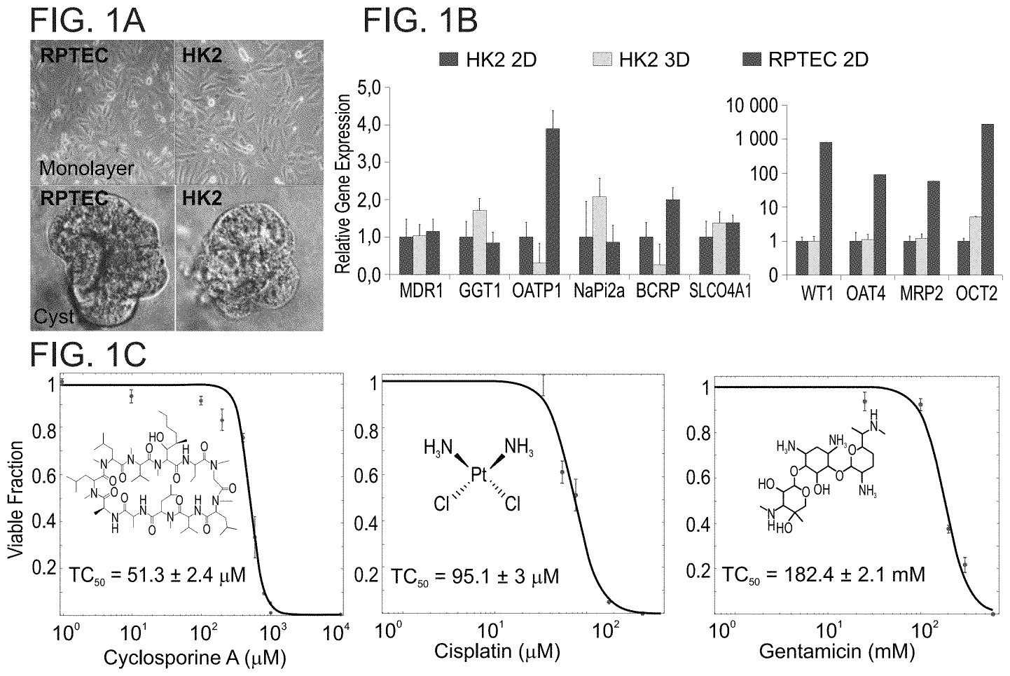

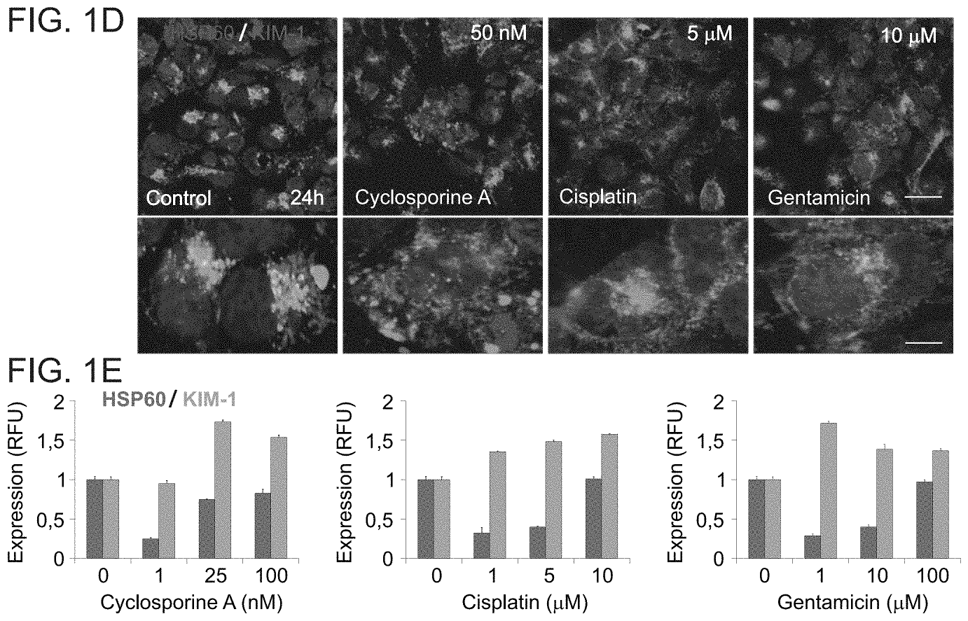

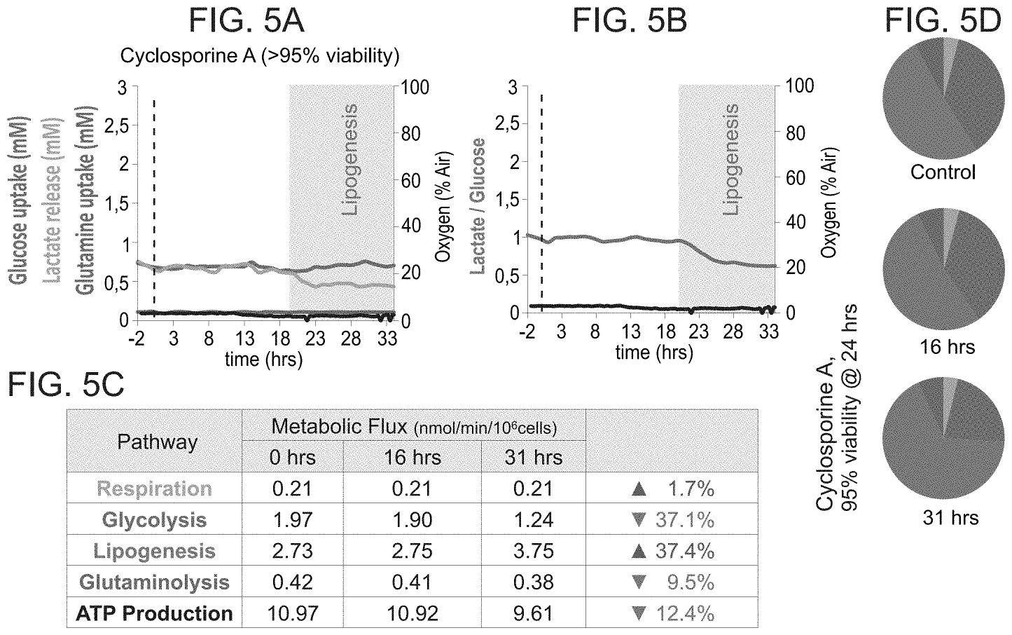

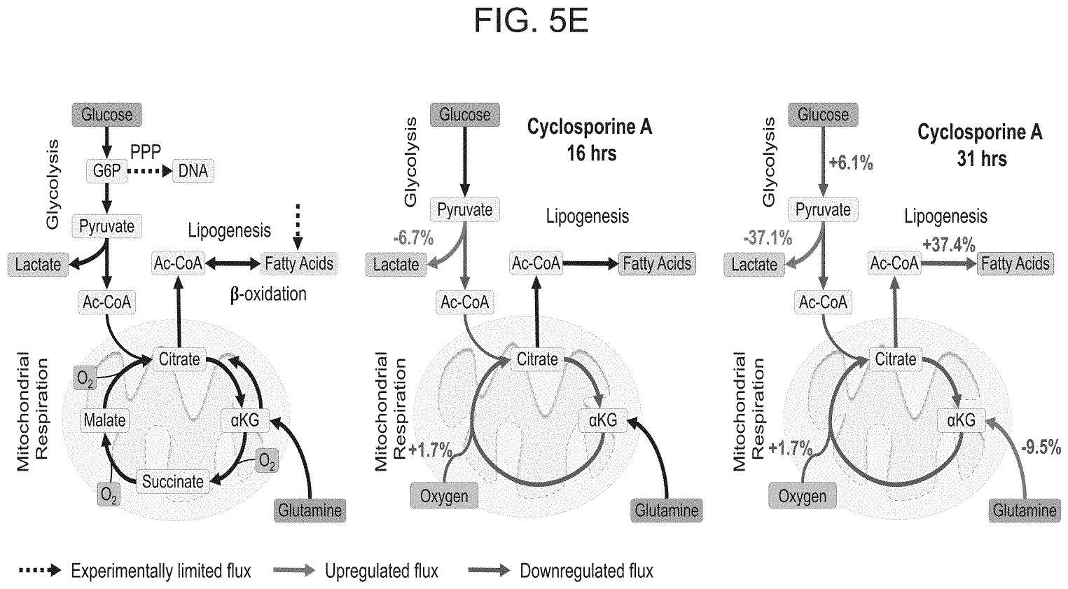

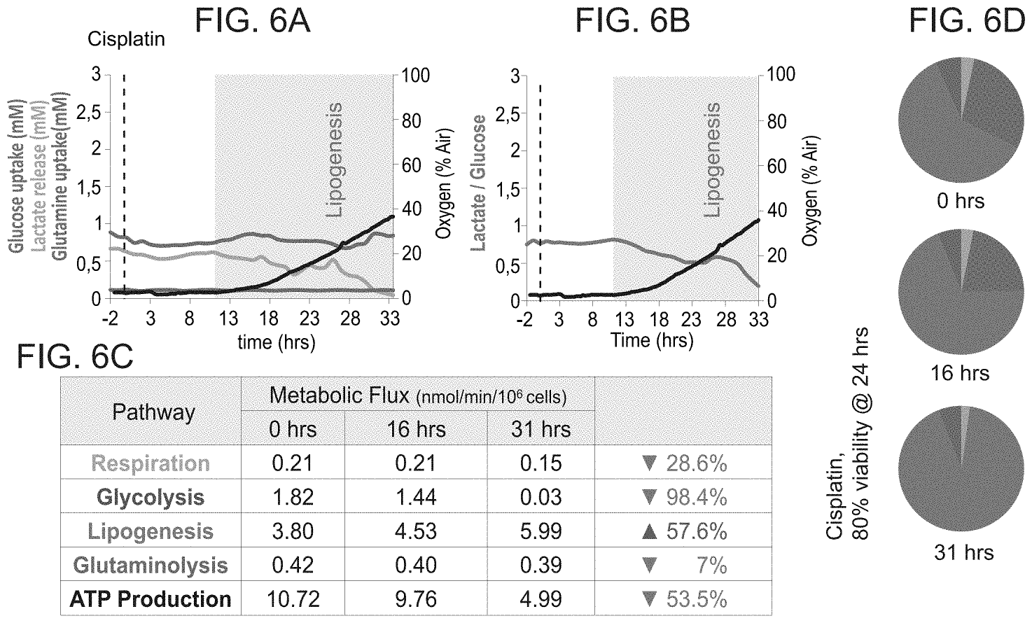

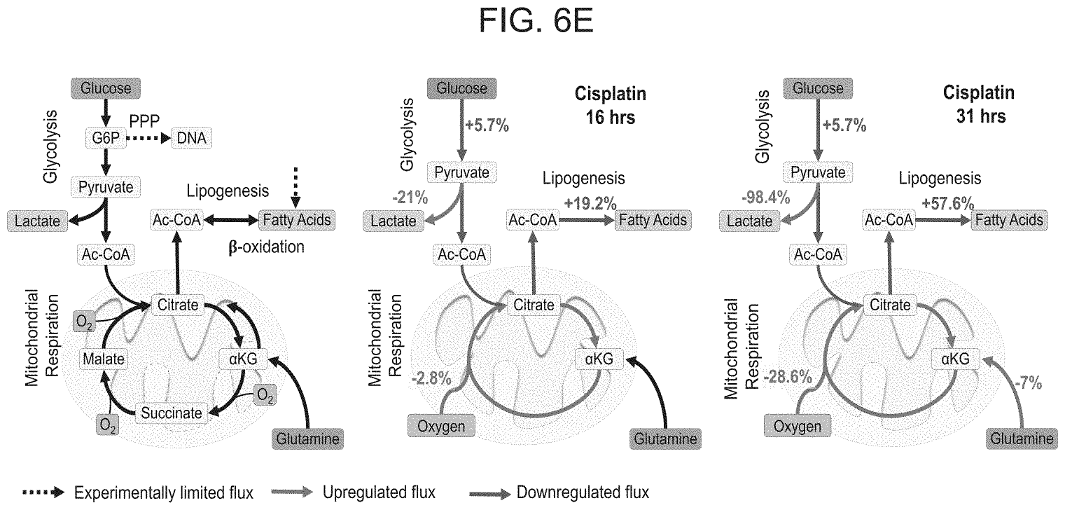

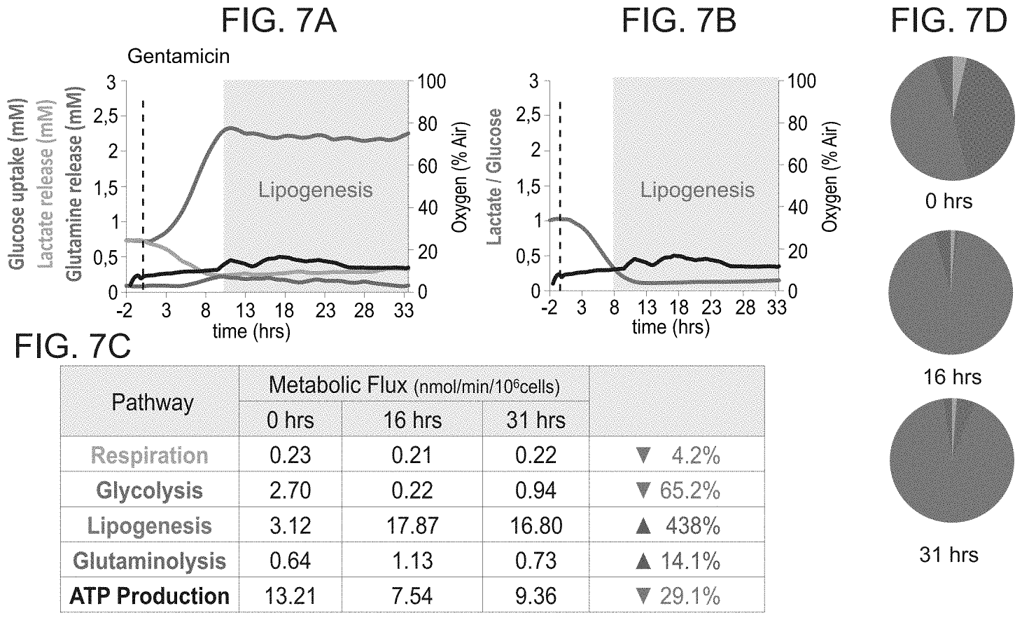

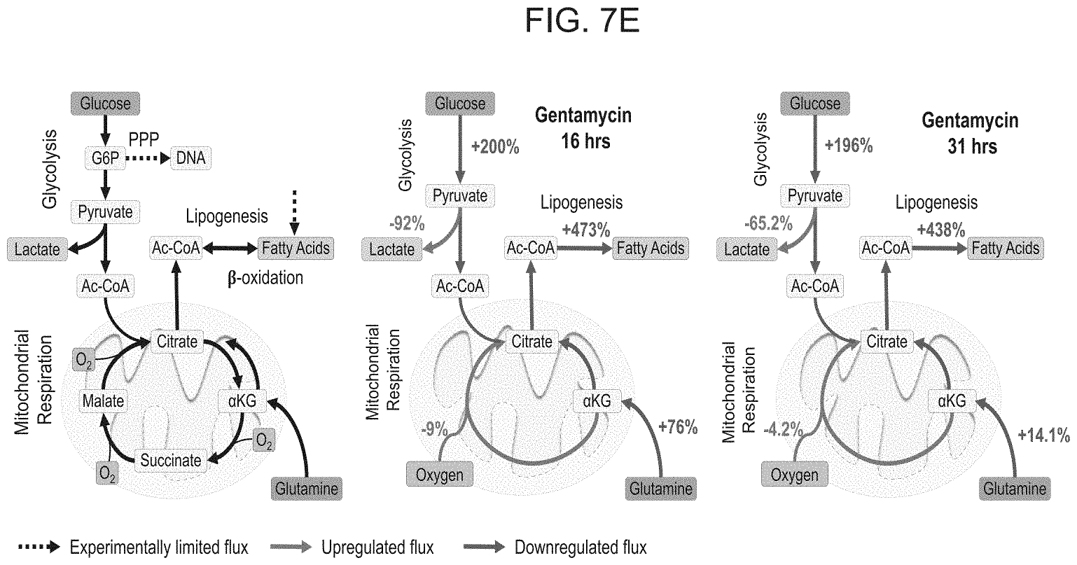

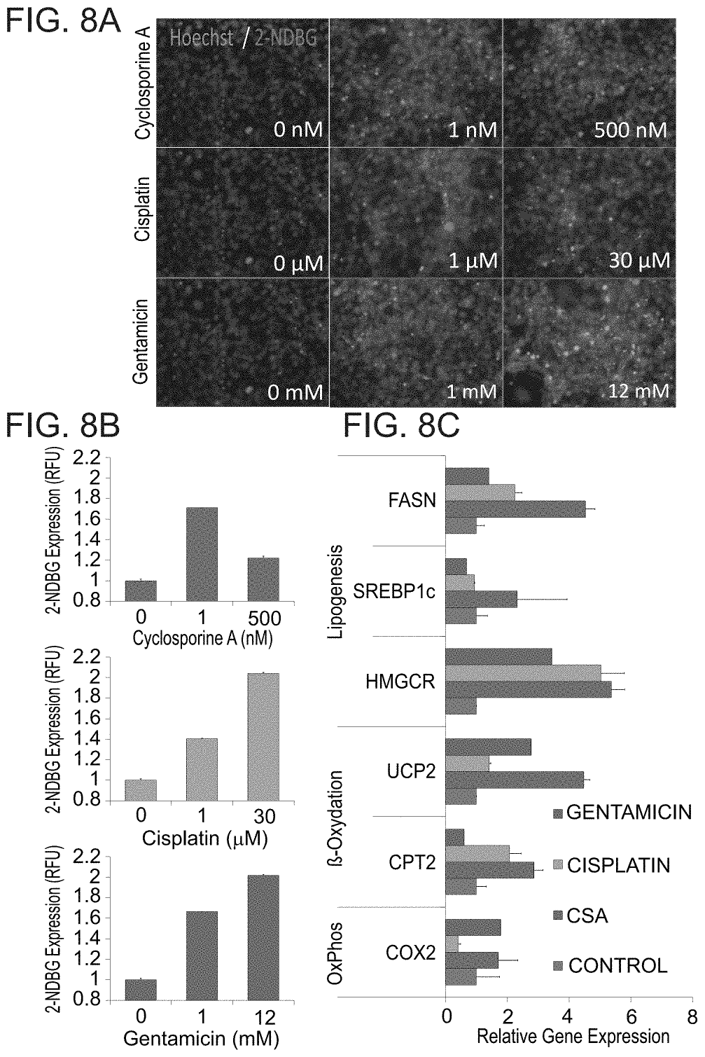

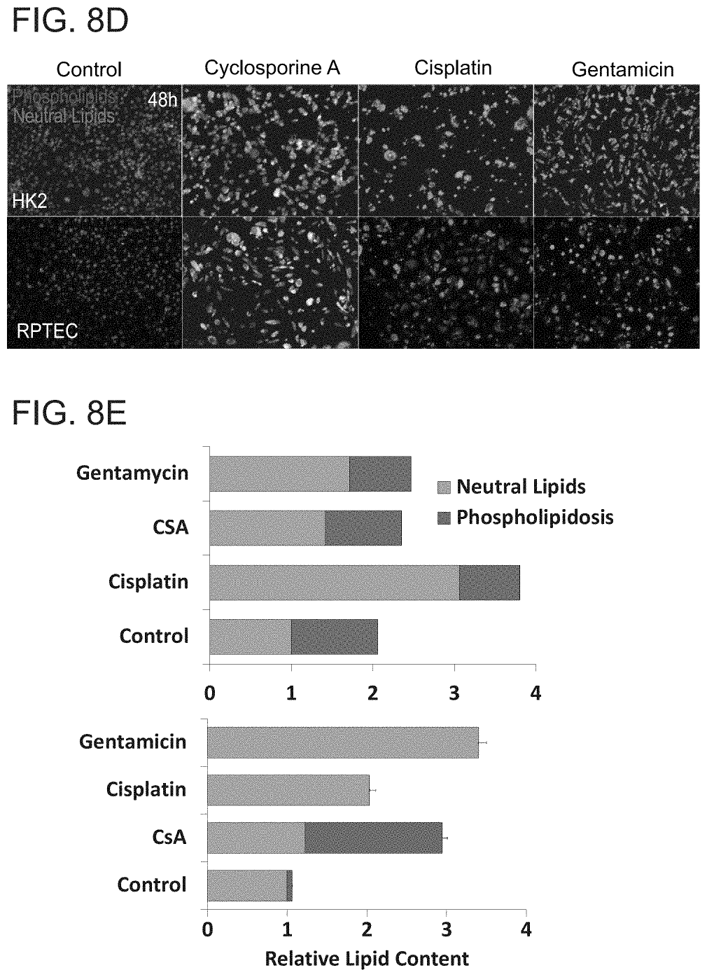

Whilst reducing the present invention to practice, the present inventors noted that concentrations below the threshold that bring about cellular damage of known nephrotoxic agents, cause an increase in the membrane expression of the Kidney Injury Molecule 1 (KIM1), a standard biomarker of drug-induced acute injury. Expression of KIM1 is correlated with an early loss of functional polarity (FIGS. 1D-E). Bioreactor studies show that cisplatin (FIGS. 6A-E), gentamicin (FIGS. 7A-E) and Cyclosporine A (FIGS. 5A-E), all known nephrotoxic drugs, significantly shift proximal tubular organoid metabolism towards lipogenesis. In addition, lipid staining showed major lipid accumulation in both HK-2s and RPTEC monolayers, showing features of phospholipidosis (FIG. 8D).

The present inventors further provide evidence that the underlying mechanism of the increase in lipid storage caused by the nephrotoxic agents is glucose accumulation. This happens upon immediate exposure to the nephrotoxic agents, and at a concentration which is a thousand-fold lower than the concentration that brings about mitochondrial stress (FIGS. 8A-E). This excess of glucose is due to the loss of polarity preventing the main glucose transporters in proximal tubules cells from performing their functions properly.

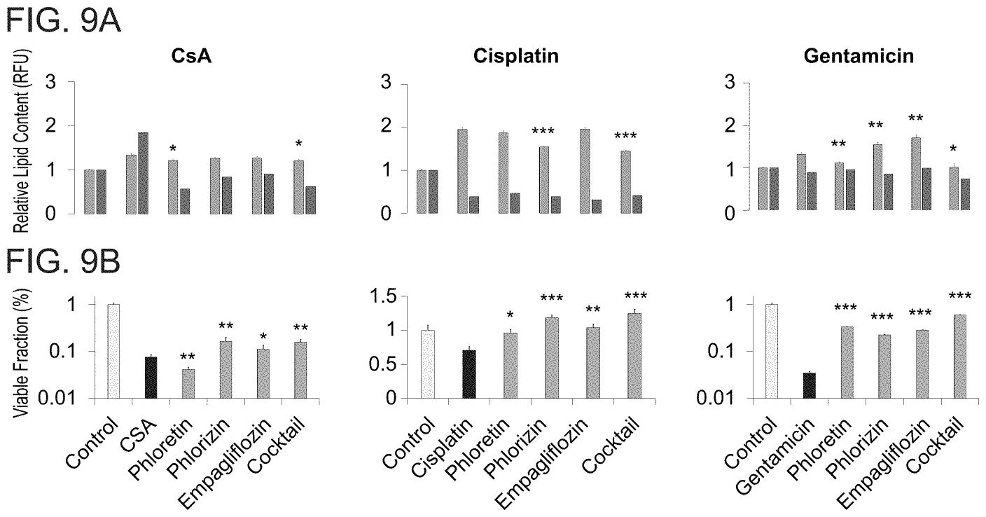

The present inventors further show that decreasing glucose uptake using Phloretin (which blocks the apicobasal shuttling of GLUT2), Phlorizin (an SGLT1 inhibitor) or Empagliflozin, (an SGLT2 inhibitor) significantly alleviates lipotoxicity and restores cell viability for all cultures treated with the three nephrotoxic compounds (FIGS. 9A-B).

Finally, the present inventors have validated the above described mechanism with clinical evidence. A retrospective clinical study was carried out to collect data from blood and urine from patients taking either Cyclosporine A or Cisplatin alone or in combination with an SGLT2 inhibitor.

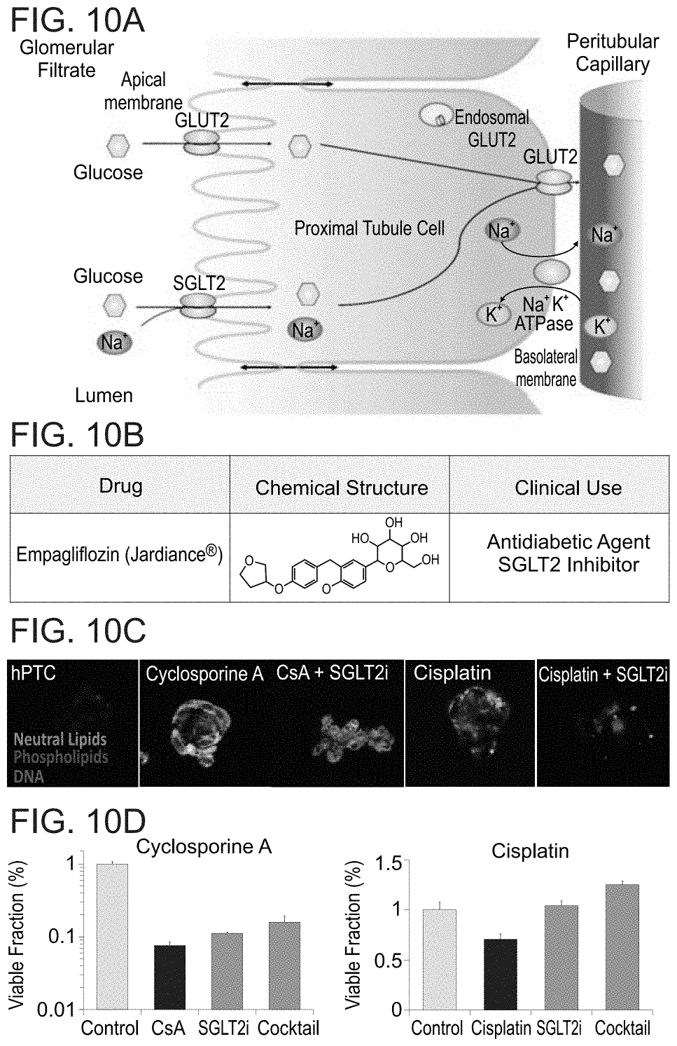

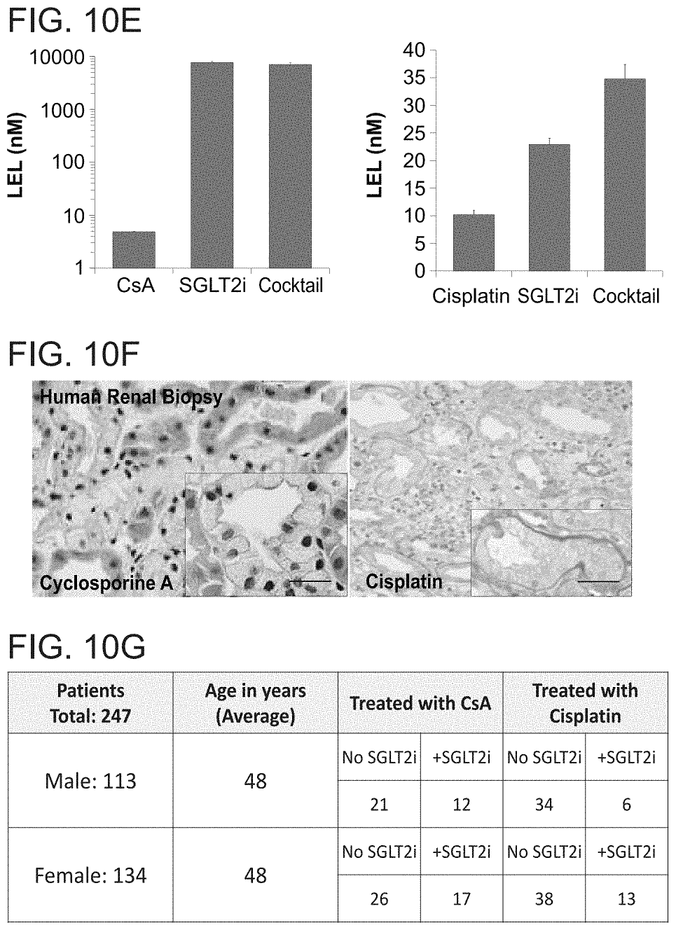

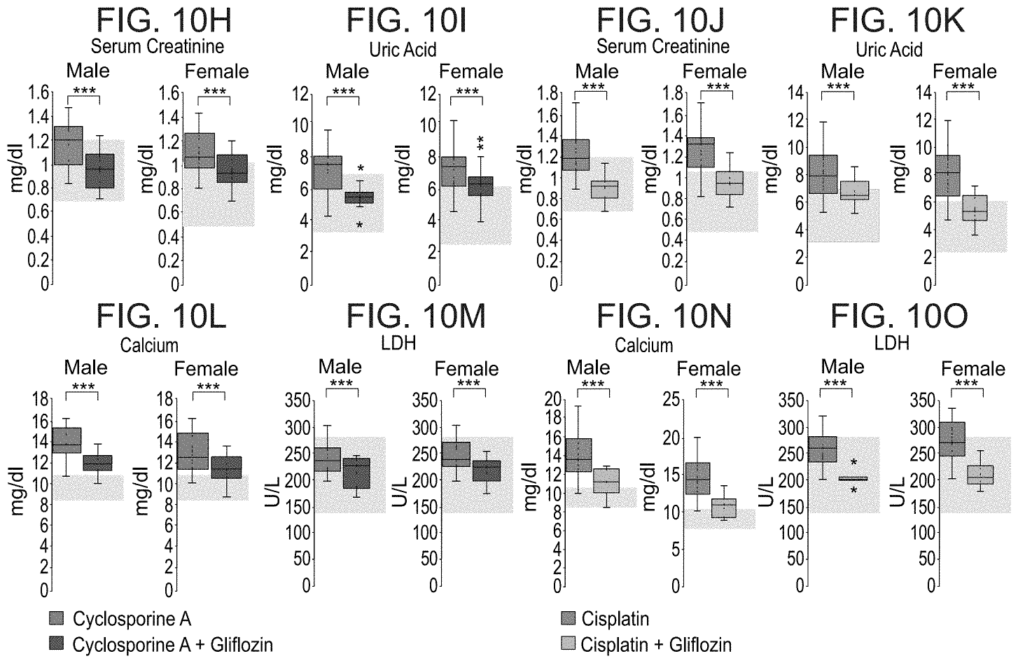

Lactate Dehydrogenase (LDH), a marker for cellular damage returned to physiological levels in patients taking Cyclosporine A or Cisplatin in combination with the SGLT2 inhibitor (FIG. 10M-O). The same phenomenon was observed for serum creatinine (FIG. 10H-J), uric acid (FIG. 10I-K) and serum calcium (FIG. 10L-N), suggesting that SGLT2 inhibitors alleviate drug-induced nephrotoxicity.

Thus, according to a first aspect of the present invention there is provided an isolated organoid comprising mature, polarized kidney epithelial cells and endothelial cells, and wherein the organoid comprises three-dimensional longitudinal tubules having at least two openings, each organoid having at least one central lumen, wherein less than 50% of the cells of the organoid express a fetal marker.

As used herein the term “organoid” refers to an artificial three-dimensional aggregate of live cells of at least two cell types. The organoid of this aspect of the present invention is generated in-vitro as further described herein below.

The organoids are typically between 100-2000 μm in diameter (for example between 200-1000 μm in diameter and may comprise between about 500-100,000 cells (for example between 1,000 and 75,000 cells).

In one embodiment, the organoid comprises only human cells.

In another embodiment, the organoid comprises non-human cells.

In one embodiment, the organoid comprises at least one epithelial lined tubule (each tubule having at least two openings). The tubules are surrounded by endothelial vessels.

According to some embodiments of the invention, the organoid can carry out at least one function of a kidney, for example glucose reabsorption.

The organoids of this aspect of the present invention comprise at least one central lumen, at least two central lumens, at least three central lumens or more.

The organoid of this aspect of the present invention is typically generated from mature human renal cells and as such does not express fetal markers.

In one embodiment, the organoid comprises mature polarized human kidney cells.

Preferably, the polarized cells of the organoid comprise epithelial cells. For example the apical surface of epithelial cells which face the tubular lumens are different in protein and lipid composition to the basolateral surface of the cells.

Preferably, less than 50% of the cells of the organoid express a fetal marker, less than 40% of the cells of the organoid express a fetal marker, less than 30% of the cells of the organoid express a fetal marker, less than 20% of the cells of the organoid express a fetal marker, less than 10% of the cells of the organoid express a fetal marker, as measured by immunohistochemistry and/or RT-PCR. In one embodiment, none of the cells of the organoid express a fetal marker. In yet another embodiment, the cells of the instant organoid express at least 10% less, 20% less, at least 30% less, at least 40% less or even at least 50% less fetal marker than a kidney organoid which is generated from non-mature cells such as embryonic stem cells, as measured under identical conditions by immunohistochemistry and/or RT-PCR.

An example of a fetal marker which is not expressed in the organoids disclosed herein is KSP (CDH16; Cadherin-16). Additional examples include OSR1 (Protein odd-skipped-related 1), WT1 (Wilms tumor 1), GDNF (Glial cell line-derived neurotrophic factor), CITED1 (Cbp/p300-interacting transactivator 1), HOXD11 (Homeobox protein Hox-D11), Wnt4 (wingless-type MMTV integration site family, member 4), Lhx1 (Lim1; LIM homeobox protein 1), Nr2f2 (COUP-TFII; nuclear receptor subfamily 2, group F, member 2) and MUC1 (mucin 1, cell surface associated).

Preferably, the organoid of this aspect of the present invention is not generated from pluripotent stem cells (e.g. embryonic stem cells) and/or renal progenitor cells.

Exemplary cells which may be used to generate the organoid disclosed herein include, but are not limited to Human Kidney-2 cells (HK-2), primary Renal Proximal Tubule Epithelial Cells (RPTEC), Human embryonic kidney 293 (HEK293) and primary kidney podocyte cells (NhKP).

The organoid of this aspect of the present invention typically expresses markers of mature cells, as measured by immunohistochemistry and/or RT-PCR. Exemplary markers expressed by the organoid include, but are not limited to ALPI (alkaline phosphatase, intestinal), Aqp1 (aquaporin 1), Cldn10 (claudin 10), Cldn11 (Osp; claudin 11), Cldn2 (claudin 2), DPP4 (DPPIV; dipeptidyl peptidase 4), Enpep (Aminopeptidase A; glutamyl aminopeptidase), GGCT (gamma-glutamylcyclotransferase), LAP3 (LAP; leucine aminopeptidase 3), MME (CD10; membrane metalloendopeptidas), Slc36a2 (solute carrier family 36 [proton/amino acid symporter], member 2), SLC5A1 (Na/Gluc1; solute carrier family 5 member 1), Slc6a18 (solute carrier family 6 [neurotransmitter transporter], member 18), Slc6a19 (solute carrier family 6 [neurotransmitter transporter], member 19), Slc6a20a (solute carrier family 6 [neurotransmitter transporter], member 20A), Slc6a20b (solute carrier family 6 [neurotransmitter transporter], member 20B).

The organoid of this aspect of the present invention may express at least one, two, three, four, five, six, seven, eight, nine or more of the above mentioned markers.

The diameter of a tubules comprised in the organoid of the present invention is typically between about 10 to 200 microns (e.g. between 50-100 microns in diameter).

The length of the tubules comprised in the organoid of the present invention is typically between 100-1000 microns (e.g. between 200-600 microns).

The organoid of the present invention may be vascularized or non-vascularized.

As used herein, the term “vascularizes organoid” refers to formation of at least a part of a 3D blood vessel network around the organoid. Typically, the blood vessel network is comprised of endothelial cells. The vasculature may be at any stage of formation as long as it comprises at least one 3D endothelial structure. Examples of 3D endothelial structures include, but are not limited to tube-like structures, preferable those comprising a lumen.

The organoids disclosed herein may be embedded with at least one microsensor for oxygen monitoring (e.g. real-time oxygen monitoring).

The microsensors are typically capable of measuring oxygen uptake (or consumption) of the cells.

In one embodiment, the microsensors are lifetime-based luminescence-quenching (LBLQ) microparticles or nanoparticles. The microparticles or nanoparticles are optionally and preferably used for measuring oxygen by determining their phase modulation. The advantage of using microparticles or nanoparticles as an oxygen sensor is that such oxygen measurement can be done without calibrating the number of cells and there is no need to operate in tiny volumes.

Microparticles and nanoparticles useful as oxygen sensors suitable for the present embodiments are found in U.S. Published Application No. 20150268224, published on Sep. 24, 2015, the contents of which are hereby incorporated by reference.

In one embodiment, the microsensor is a nanoparticle or microparticle (e.g. about 50 microns in diameter) loaded with a ruthenium-based dye.

The ratio of microsensor:cells comprised in the cell suspension which is used to generate the organoid is typically between 0.5-4 mg (milligram) per milliliter of cell suspension.

Other exemplary microsensors which can be used to measure oxygen uptake include but are not limited to 50 micrometer-diameter polystyrene microbeads loaded with ruthenium-phenanthroline-based phosphorescence dye such as CPOx-50-RuP (Colibri Photonics) or 200 nm-diameter beads OXNANO (Pyro Science).

To generate the organoids of the present invention renal cells such as Human Kidney-2 cells (HK-2) or primary Renal Proximal Tubule Epithelial Cells (RPTEC) are cultured at a density of about 0.5-10×10⁴ (e.g. 7.5×10⁴ cells) per 1.5 mm well. Preferably, the cells are cultured on an extracellular matrix (e.g., Matrigel® or laminin) in the presence of a culture medium. The cells are cultured under conditions that promote organoid formation (e.g. at 37° C. in a humidified incubator with about 5% CO₂ for 10-24 hours).

According to a particular embodiment, the organoids are cultured in a bioreactor which is continuously perfused with cell culture medium (for example a microfluidic array). The microfluidic array may comprise a plurality of wells for culturing the organoids.

A perfusion element may be used for generating, in a controlled manner, a flow of a perfusion medium onto the array, wherein the flow is controlled so as to ensure continuous perfusion. During the flow, signals indicative of one or more physiological parameters may be collected from the array. The signals can be recorded on a computer readable medium, preferably a non-transitory computer readable medium. Alternatively or additionally, the signals can be analyzed to determine one or more physiological parameters that are characteristic of the cells of the organoid on the array.

According to embodiments of the invention there is provided a multi-well plate comprising an array of wells each containing a distinct organoids therein, wherein a size of each organoid is within less than 20% or less than 15% or less than 10% from an average size of all organoids occupying the array.

According to some embodiments of the invention, the organoids present in the wells of the multi-well plate are homogenous in terms of size and/or cell number.

According to some embodiments of the invention, each well has a single (distinct organoid), wherein all organoids are homogenous.

When the organoid is cultured in a microfluidic array, the microwell may comprise an insert so as to protect the cells from the negative effects of shear force. The insert may be fabricated from materials known in the art to be compatible with tissue culturing—for example polydimethylsiloxane (PDMS), glass, Poly(methyl methacrylate) (PMMA), Cyclic olefin copolymer (COC), Polycarbonate, or Polystyrene The array may further comprise a temperature sensor, a glucose sensor and/or a lactate sensor and/or a glutamine sensor.

The lactate and/or glucose sensor may be electrochemical—e.g. allowing for amperometric measurement of lactate and/or glucose.

In one embodiment pH of the medium is measured as a surrogate for lactate measurement.

The array of the present embodiments may further comprise at least one of: an electronic control circuit for signal modulation and read-out, an light source (e.g., LED) for excitation (e.g., of the oxygen sensing particles), an optical filter set (e.g., 531/40, 555, 607/70 nm) and a detector unit containing a photomultiplier (PMT). One skilled in the art would appreciate that the sensing particles can be excited by various wave lengths depending on the specific sensing particles used. Accordingly, emission may be read at various wave lengths as well.

Preferably the array has a three-electrode design in which the counter and reference electrodes are separated. The reference electrode is used to measure the working electrode potential without passing current through it, while the counter electrode closes a circuit, allowing current to pass.

The organoids disclosed herein may be useful for determining the toxic effect of a candidate agent on the kidney.

Thus, according to another aspect of the present invention there is provided a method of determining the toxic effect of a candidate agent on the kidney, the method comprising:

(i) providing the organoid described herein (wherein the organoid comprises at least one microsensor for oxygen monitoring;

(ii) culturing the organoid under physiological conditions in the presence of said candidate agent; and

(iii) performing real-time measurements of oxygen consumption of said organoid, wherein a decrease in oxygen consumption of said organoid in the presence of said candidate agent as compared to the oxygen consumption of said organoid in the absence of said candidate agent is indicative that the candidate agent has a toxic effect on the kidney.

Examples of agents that may be tested include, but are not limited to therapeutic agents, diagnostic agents, imaging agents such (e.g. dyes), food, cosmetics, and agents suspected of having environmental nephrotoxic effect.

Exemplary measurements of oxygen consumption that can be carried out on the organoids include at least one of the following:

a. determination of the percentage of the reduction of the oxygen consumption caused by the agent;

b. determination of the duration of the exposure to the candidate agent that causes a reduction (for example a 10% reduction, a 20% reduction, a 30% reduction, a 40% reduction, a 50% reduction etc. in the oxygen consumption; and

c. determination of the concentration of the candidate agent that causes a 10% reduction, a 20% reduction, a 30% reduction, a 40% reduction, a 50% reduction of the oxygen consumption.

In a particular embodiment wherein the oxygen sensing particle is a ruthenium-phenanthroline-based particle, the particles are excited by 532 nm and a 605 nm emission is read, so as to measure phosphorescence decay, substantially in real time.

The agents which are being tested are preferably present at a concentration that causes less than 50%, 40%, 30%, 20%, 10% of the cells in the organoid to die in 24 hours.

As well as measuring the amount of oxygen in the system outflow (so as to determine oxygen consumption of the cells), the present inventors envisage measuring lactate in the system outflow (so as to determine lactate production of the cells) and/or measuring glucose in the system outflow (so as to determine glucose usage of the cells) and/or measuring glutamine in the system outflow (so as to determine glutamine production of the cells).

Glutamine, like glucose and lactate, can also be measured electrochemically. For example, glutaminase and glutamate oxidase enzymes may be immobilized in a membrane. Glutamine is transformed to glutamic acid by glutaminase, and the glutamic acid is transformed by glutamate oxidase to form a detectable reaction product using amperometric or potentiometric sensor. Sensors can be purchased from Innovative Sensor Technologies (Las Vegas, Nev.). Other methods of measuring glutamine production are described in WO1988010424 A1, U.S. Pat. No. 4,780,191.

Usually, glutaminase and glutamate oxidase enzymes are immobilized in a membrane. Glutamine is transformed to glutamic acid by glutaminase, and the glutamic acid is transformed by glutamate oxidase to form a detectable reaction product using amperometric or potentiometric sensor. Sensors can be purchased from Innovative Sensor Technologies (Las Vegas, Nev.).

According to a particular embodiment, the constituents of the perfusion medium are such that at least one metabolic pathway of the organoid is eliminated. Thus for example the perfusion medium may be deficient in at least one nutrient type.

It will be appreciated that the term “deficient” does not necessarily mean that the medium is totally devoid of that constituent, but that it may be present in limited amounts such that the at least one metabolic pathway of the cells of the organoid is eliminated. Thus, for example trace amounts of the constituent may be present in the proliferation medium.

Elimination of a pathway (i.e. bypassing of a pathway, or shunting away from the use of the pathway) also does not have to be total. In one embodiment, utilization of that pathway is at least 10 times, 20 times, 50 times or even 100 times lower than an alternate pathway which generates the same end-product and/or which uses the same starting material.

It will be appreciated that the term “elimination of a pathway” may refer to the bypassing of, or shunting away from, only one or both directions of a pathway.

Elimination of a pathway is important such that when metabolic flux is calculated this pathway (or pathway in one particular direction) can be neglected.

The eliminated pathway includes for example the lipid oxidation pathway, glycolysis, glutaminolysis, urea cycle, lipogenesis, cholesterol synthesis, mevalonate pathway, and the anaplerotic reactions replenishing the TCA cycle (e.g. valine, isoleucine).

Thus for example in the case where the medium is lipid deficient or limited in lipids, the amount of lipids in the medium is such that fatty acid uptake is at least 10 fold, 20 fold, 50 fold or even 100 fold lower than glucose uptake in the cell.

Examples of nutrient types which may be deficient include, but are not limited to a fatty acid, triglyceride, cholesterol, monosaccharide, pyruvate, glycerol and an amino acid.

For example, if pyruvate and glycerol are deficient in the perfusion medium, the metabolic pathways into glycolysis are eliminated. This is useful for determining the amount of glycolysis of the cell.

For example if lipids such as fatty acids and triglycerides are deficient in the perfusion medium, the lipid oxidation pathway is eliminated. This is useful for determining the amount of glycolysis, oxidative phosphorylation, and/or glutaminolysis of the cells.

For example, the constituents of the perfusion medium can be selected which induce or inhibit glycolytic flux. Then, measurements of oxygen, glucose and/or lactate can be made as further detailed hereinabove and used for determining glycolytic capacity, glycolytic reserve and/or non-glycolytic acidification. In a representative example, which is not to be considered as limiting, non-glycolytic acidification is determined before perfusion of the environment altering constituents, glycolytic capacity can be determined following perfusion with saturating concentration of glucose, which is used by the cells through the glycolytic pathway to produce ATP, NADH, water, and protons, and glycolytic reserve is determined following perfusion with glycolysis inhibitor, such as, but not limited to, 2-deoxyglucose, which inhibits glycolysis by binding to hexokinase.

Exemplary metabolic flux (e.g. the rate of turnover of molecules through a particular metabolic pathway), that may be calculated using the system described herein are set forth in Table 1 herein below.

TABLE 1
Metabolic flux
Glucose + Pᵢ [symbol] Glucose 6-P + H₂0
Glucose 6-P [symbol] Fructose 6-P
Fructose 6-P + Pi [symbol] Fructose 1,6-P₂ + H₂0
Fructose 1,6-P₂ [symbol] 2 Glyceraldehyde 3-P
Glyceraldehyde 3-P + NAD⁺ + Pᵢ + ADP [symbol]
Phosphoenolpyruvate + NADH + H⁺ + ATP
Phosphoenolpyruvate + ADP → Pyruvate + ATP
Pyruvate + CoA + NAD⁺ → Acetyl-CoA + CO₂ + NADH
Lactate + NAD⁺ [symbol] Pyruvate + NADH + H⁺
Acetyl-CoA + Oxaloacetate + H₂O → Citrate + CoA + H⁺
Citrate + NAD + [symbol] 2-oxo-gluterate + NADH + CO₂
2-oxo-gluterate + NAD⁺ + CoA → Succinyl-CoA + NADH + CO₂ + H⁺
Succinyl-CoA + Pᵢ + GDH + FDH [symbol] Fumarate + GTP + FADH₂ + CoA
Fumarate + H₂O [symbol] Malate
Malate + NAD + [symbol] Oxaloacetate + NADH + H⁺
Ornithine + CO₂ + NH₄⁺ + 2 ATP + H₂O [symbol] Citrulline + 2 ATP + 2Pᵢ + 3H⁺
Citrulline + Aspartate + ATP → Arginine + Fumarate + AMP + PPᵢ
Arginine uptake
Ammonia production
Ornithine Output
Alanine + 0.5 NAD⁺ + 0.05 NADP⁺ + H₂O →
Pyruvate + NH₄⁺ + 0.5 NADH + 0.5 NADPH + H⁺
Alanine Uptake
Serine → Pyruvate + NH₄⁺
Serine Uptake
Cysteine + 0.5 NAD⁺ + 0.5 NADP⁺ + H₂O + SO₃ ²⁻→
Pyruvate + Thiosulfate + NH₄⁺ + 0.5 NADH + 0.5 NADPH + H⁺
Cysteine Uptake
Threonine + NAD⁺ → Glycine + Acetyl-CoA + NADH
Glycine + NAD⁺ + H₄folate [symbol] N⁵, N¹⁰-CH₂H₄folate + NADH + CO₂ + NH₄⁺ + H⁺
Glycine Uptake
Tryptophan + 3 H₂O + 3 O₂ + CoA + 3 NAD⁺ + FAD →
3CO₂ + FADH₂ + 3NADH + 4H⁺ + NH₄⁺ + Acetoacetatyl-CoA
Propionyl-CoA + CO₂ + ATP → Succinyl-CoA + AMP + PPᵢ
Lysine + 3H₂O + 5 NAD⁺ + FAD + CoA →
2 NH₄⁺ + 5 NADH + 5 H⁺ + FADH₂ + 2 CO₂ + Acetoacetatyl-CoA
Phenylalanine + H₄biopterin + O₂ → Tyrosine + H₂biopterin + H₂O
Tyrosine + 0.5 NAD⁺ + 0.5 NADP⁺ + H₂O + 2 O₂ →
NH₄⁺ + CO₂ + 0.5 NADH + 0.5 NADPH + H⁺ + Fumarate + Acetoacetate
Tyrosine Uptake
Glutamate + 0.5 NAD⁺ + 0.5 NADP⁺ + H₂O [symbol]
NH₄⁺ + 2-oxo-gluterate + 0.5 NADH + 0.5 NADPH + H⁺
Glutamate Uptake
Glutamine + H₂O → Glutamate + NH₄⁺
Ornithine + NAD⁺ + NADP⁺ + H₂O → Glutamate + NH₄⁺ + NADH + NADPH + H⁺
Proline + 0.5 O₂ + 0.5 NAD⁺ + 0.5 NADP⁺ →
Glutamate + 0.5 NADH + 0.5 NADPH + H⁺
Histidine + H₄folate + 2H₂O → NH₄⁺ + N5-formiminoH₄folate + Glutamate
Methionine + ATP + Serine + NAD⁺ + CoA →
PPᵢ + Pᵢ + Adensosine + Cysteine + NADH + Propionyl-CoA + CO₂ + NH₄⁺
Aspartate + 0.5 NAD⁺ + 0.5 NADP⁺ + H₂O [symbol]
Oxaloacetate + NH₄⁺ + 0.5 NADH + 0.5 NADPH + H⁺
Aspartate Uptake
Asparagine + H₂O → Aspartate + NH₄⁺
8 Acetyl-CoA + 7 ATP + 14 NADPH + 14 H⁺ →
Palmitate + 8 CoA + 6 H₂O + 7 ADP + 7Pᵢ + 14 NADP⁺
2 Acetyl-CoA [symbol] Acetoacetyl-CoA + CoA
Acetoacetyl-CoA + H₂O → Acetoacetate + CoA
Acetoacetate Output
Acetoacetate + NADH + H⁺ [symbol] β-hydroxybutyrate + NAD⁺
NADH + H⁺ + 0.5 O₂ + 3 ADP → NAD⁺ + H₂O + 3 ATP
FADH₂ + 0.5 O₂ + 2 ADP → FAD + H₂O + 2 ATP
O₂ Uptake
Glucose 6-P + 12 NADP⁺ + 7 H₂O → 6 CO₂ + 12 NADPH + 12 H⁺ + Pᵢ
Valine + 0.5 NADP⁺ + CoA + 2 H₂O + 3.5 NAD⁺ + FAD →
NH₄⁺ + Propionyl-CoA + 3.5 NADH + 0.5 NADPH + 3 H⁺ + FADH₂ + 2 CO₂
Isoleucine + 0.5 NADP⁺ + H₂O + 2.5 NAD⁺ + FAD + 2 CoA →
NH₄⁺ + Propionyl-CoA + Acetyl-CoA + 2.5 NADH + 0.5 NADPH + 3H⁺ +
FADH₂ + CO₂
Leucine + 0.5 NADP⁺ + H₂O + 1.5 NAD⁺ + FAD + ATP + CoA →
NH₄⁺ + 1.5 NADH + 0.5 NADPH + 2 H⁺ + FADH₂ + ADP + Pᵢ +
Acetoacetate + Acetyl-CoA
Threonine uptake
Lysine Uptake
Phenylalanine Uptake
Glutamine Uptake
Proline Uptake
Histidine Uptake
Methionine Uptake
Asparagine Uptake
Valine Uptake
Isoleucine Uptake
Leucine Uptake
Protein Synthesis
Triglyceride [symbol] Glycerol + 3 Palmitate
Triglyceride Uptake
Glycerol Uptake
Palmitate Uptake
Glucose-6-P + UTP + H₂O [symbol] Glycogen + 2 Pᵢ + UDP
Glycerol + NAD⁺ [symbol] Glyceraldehyde 3-P + NADH + H⁺
18 Acetyl-CoA + 25 NADPH + NADH + 26 H⁺ + 18 ATP + 11 O₂ →
Cholesterol + 25 NADP⁺ + NAD⁺ + 18 ADP + 6 Pᵢ + PPᵢ + 8 CO₂ +
6 H₂O + 18 CoA + HCOOH
Cholesterol + 5 NADPH + H⁺ + 3 O₂ + ATP + 2 CoA + FAD [symbol]
Choloyl-CoA + 5 NADP⁺ + 2 H₂O + ADP + PPᵢ + FADH₂ + Propionil-CoA
Cholesterol Output
CO₂ output

Using the organoids described herein, the present inventors uncovered that drug-induced nephrotoxicity is caused by glucose build-up in renal cells causing lipid accumulation in the tubules, especially in the proximal tubule cells, which leads to lipotoxicity in renal tissues. The excess of glucose (which occurs at drug concentrations that are approximately one thousand fold smaller than those previously considered toxic) was found to be due to the loss of polarity of proximal tubule cells, preventing the main glucose transporters in these cells to perform their functions properly.

The present inventors thus propose that drug-induced nephrotoxicity can be reduced or even eliminated by the prevention, elimination or reduction of lipid accumulation in the renal tis sue.

Thus, according to another aspect of the present invention there is provided a method for reducing renal tissue toxicity in a subject caused by a kidney damaging agent, the method comprising administering to the subject:

(i) a kidney damaging agent; and

(ii) an agent that protects the polarity of proximal tubule cells from the toxic effect of said kidney damaging agent, thereby reducing renal toxicity in the subject, with the proviso that when the kidney damaging agent is glucosfamide, said agent that protects the polarity of proximal tubule cells is not an inhibitor of SGLT2.

According to another aspect of the present invention there is provided a method for reducing renal tissue toxicity in a subject caused by a kidney damaging agent, the method comprising administering to the subject:

(i) a kidney damaging agent; and

(ii) an inhibitor of glucose reabsorption, thereby reducing renal toxicity in the subject, with the proviso that when the kidney damaging agent is glucosfamide, the inhibitor of glucose reabsorption is not an inhibitor of SGLT2.

According to another aspect of the present invention there is provided a method for reducing renal tissue toxicity in a subject caused by a kidney damaging agent, the method comprising administering to the subject:

(i) a kidney damaging agent; and

(ii) an agent that causes a decrease in lipid accumulation in renal tissue of the subject, thereby reducing renal toxicity in the subject, with the proviso that when the kidney damaging agent is glucosfamide, said agent that causes a decrease in lipid accumulation is not an inhibitor of SGLT2.

As used herein, the term “kidney damaging agent” refers to a therapeutic agent, diagnostic agent, imaging agent such (e.g. dyes), food, a cosmetic, which when used at clinically acceptable doses causes unwanted damage to the kidney. The kidney damaging agent typically has a primary function that brings about a diagnostic or therapeutic effect and has a negative side-effect of causing damage to renal tissue.

In one embodiment, the kidney damaging agent is one that disrupts the polarity of the polarity of proximal tubule epithelial cells.

The term “renal tissue” refers to any tissue of the kidney. In one embodiment the renal tissue comprises renal tubules, especially proximal tubule cells.

Examples of therapeutic agents which are known to damage the kidney are provided in Table 2.

TABLE 2

| Medication | Drug category | Renal toxicity |
|---|---|---|
| Acetaminophen | Non-narcotic analgesic | Chronic interstitial nephritis, acute |
| | | tubular necrosis |
| Acetazolamide | Carbonic-anhydrase | Proximal renal tubular acidosis |
| | inhibitor | |
| Acyclovir | Antiviral | Acute interstitial nephritis, crystal |
| | | nephropathy |
| Allopurinol | Hypouricemic agent | Acute interstitial nephritis |
| Aspirin | Non-narcotic analgesic | Chronic interstitial nephritis |
| Amitriptyline | Antidepressant | Rhabdomyolysis |
| Aminoglycosides | Antimicrobial | Acute tubular necrosis |
| Amphotericin B | Antifungal | Acute tubular necrosis, distal renal |
| | | tubular acidosis |
| Angiotensin-converting | Antihypertensive | Acute kidney injury |
| enzyme inhibitors (ACEI) | | |
| Angiotensin receptor | Antihypertensive | Acute kidney injury |
| blockers (ARB) | | |
| Benzodiazepines | Sedative-Hypotonic | Rhabdomyolysis |
| Beta lactams | Antimicrobial | Acute interstitial nephritis |
| Carbenicillin | Antimicrobial | Metabolic alkalosis |
| Cephalosporin | Antimicrobial | Acute tubular necrosis |
| Cholpropamide | Sulfonylureas | Hyponatremia, syndrome |
| | | inappropriate ADH secretion |
| Cimetidine | Gastrointestinal | Acute interstitial nephritis |
| Cisplatin | Antineoplastic | Chronic interstitial nephritis |
| Clopidogrel | Antiplatelet | Thrombotic miroangiopathy |
| Cocaine | Narcotic analgesic | Rhabdomyolysis |
| Contrast agents | Contrast medium | Acute tubular necrosis |
| Cortisone | Cortico steroid | Metabolic alkalosis, hypertension |
| Cyclophosphamide | Antineoplastic | Hemorrhagic cystitis |
| Cyclosporine | Immunosuppressive | Acute tubular necrosis, chronic |
| | | interstitial nephritis, thrombotic |
| | | microangiopathy |
| D-penicillamine | Antirheumatic | Nephrotic syndrome |
| Diphenhydramine | Antihistamine | Rhabdomyolysis |
| Furosemide | Loop diuretic | Acute interstitial nephritis |
| Ganciclovir | Antiviral | Crystal nephropathy |
| Gold Na thiomalate | Aniarthritic | Glomerulonephritis, nephrotic |
| | | syndrome |
| Haloperidol | Antipsychotic | Rhabdomyolysis |
| Indinavir | Antiviral | Acute interstitial nephritis, crystal |
| | | nephropathy |
| Interferon-alfa | Antineoplastic | Glomerulonephritis |
| Lansoprazole | Proton pump inhibitor | Acute interstitial nephritis |
| Lithium | Antipsychotic | Chronic interstitial nephritis, |
| | | glomerulonephritis, rhabdomyolysis |
| Methadone | Narcotic analgesic | Rhabdomyolysis |
| Methamphetamine | Psycho stimulant | Rhabdomyolysis |
| Methotrexate | Antineoplastic | Crystal nephropathy |
| Mitomycin-C | Antineoplastic | Thrombotic microangiopathy |
| Naproxen | Nonsteroidal anti- | Acute and chronic interstitial |
| | inflammatory | nephritis, acute tubular necrosis, |
| | | glomerulonephritis |
| Omeprazole | Proton pump inhibitor | Acute interstitial nephritis |
| Pamidronate acid | Bisphosphonate, | Glomerulonephritis |
| | osteoporosis prevention | |
| Pantoprazole | Proton pump inhibitor | Acute interstitial nephritis |
| Penicillin G | penicillin | Glomerulonephritis |
| Pentamidine | Antimicrobial | Acute tubular necrosis |
| Phenformin | Hypoglycemic | Lactic acidosis |
| Phenacetin | Non-narcotic analgesic | Chronic interstitial nephritis |
| Phenytoin | Anticonvulsant | Acute interstitial nephritis, diabetes |
| | | insipidus |
| Probenecid | Uricosuric | Crystal nephropathy, nephrotic |
| | | syndrome |
| Puromycin | Antimicrobial | Nephrotic syndrome |
| Quinine | Muscle relaxant | Thrombotic microangiopathic |
| Quinolones | Antimicrobial | Acute interstitial nephritis, crystal |
| | | nephropathy |
| Rifampin | Antimicrobial | Acute interstitial nephritis |
| Ranitidine | Gastrointestinal | Acute interstitial nephritis |
| Statins | Lipid-lowering | Rhabdomyolysis |
| Sulfonamides | Antimicrobial | Acute interstitial nephritis, crystal |
| | | nephropathy |
| Tacrolimus | Immunosuppressive | Acute tubular necrosis |
| Tetracycline | Antimicrobial | Acute tubular necrosis |
| azides | Diuretic | Acute interstitial nephritis |
| Tolbutamide | Hypoglycemic | Nephrotic syndrome |
| Vancomycine | Antimicrobial | Acute interstitial nephritis |

According to a particular embodiment, the therapeutic agent is gentamycin, cyclosporine or cisplatin.

In the case when the kidney damaging agent is a therapeutic agent, the kidney damaging agent is provided to the subject in order to treat the disease. In one embodiment, the subject is in chronic need of the therapeutic agent.

According to a specific embodiment, the subject does not have a metabolic disease including for example diabetes, atherosclerosis and lipid disorders including primary elevated cholesterol, dyslipidemic syndrome, primary elevated triglycerides, primary low-HDL syndromes, Familial hypercholesterolemia, (a genetic disorder that increases total and LDL cholesterol) and familial hypertriglyceridemia.

Preferably, the kidney damaging agent is not being used to treat an underlying kidney disease.

Thus, for example in the case of cisplatin, the subject of this aspect of the present invention has a cancer (e.g. testicular cancer, ovarian cancer, cervical cancer, breast cancer, bladder cancer, head and neck cancer, esophageal cancer, lung cancer, mesothelioma, brain tumors or neuroblastoma). In the case of cyclosporine, the subject of this aspect of the present invention may have a disease such as rheumatoid arthritis, psoriasis, Crohn's disease, or may have had an organ transplant. In the case of gentamycin, the subject of this aspect of the present invention may have a bacterial infection, including but not limited to include bone infections, endocarditis, pelvic inflammatory disease, meningitis, pneumonia, urinary tract infections, and sepsis.

As mentioned, the method of this aspect of the present invention further comprises administration of agents that protect the polarity of proximal tubule epithelial cells from the toxic effect of said kidney damaging agent.

In some embodiments, the agents cause a decrease in lipid accumulation. Such agents typically reduce the accumulation of triglyceride-rich lipid droplets and/or phospholipid rich lysosomes in the cytoplasm of the cells.

Agents that protect the polarity of proximal tubule epithelial cells from the toxic effect of said kidney damaging agent include those that inhibit glucose reabsorption, those that block lipid synthesis and those that upregulate lipid oxidation.

In one embodiment, the agent reduces lipid accumulation in renal cells to a greater extent than said agent downregulates lipid accumulation in non-intestinal cells.

In another embodiment, the agent downregulates the amount of intracellular glucose in renal cells to a greater extent than the agent downregulates the amount of glucose in non-renal cells.

According to one embodiment, the agent is an inhibitor of glucose metabolism.

In another embodiment, the agent is an inhibitor of a glycolytic enzyme, examples of which are summarized in Table 3 herein below.

TABLE 3

| Compound name | Target protein |
|---|---|
| 2-DG | Inhibits HK |
| 3-BP | Inhibits HK |
| Lonidamine | Inhibits mitochondrial HK2 |
| 3PO | Inhibits PFK2 |
| N4A, YZ9 | Inhibits PFK2 |
| PGMI-004A | Inhibits PGAM1 |
| MJE3 | Inhibits PGAM1 |
| TT-232 | Inhibits PKM2 |
| Shikonin/alkannin | Inhibits PKM2 |
| ML265 (TEPP-46) | Activates PKM2 |
| FX11 | Inhibits LDHA |
| Quinoline 3-sulfonamides | Inhibit LDHA |
| DCA | Inhibits PDK |
| 6-AN | Inhibits G6PD |
| Oxythiamine | Inhibits TKTL1 |

2-DG: 2-deoxyglucose;
3-BP: 3-bromopyruvate;
DCA: Dichloroacetate;
6-AN: 6-aminonicotinamide;
HK: Hexokinase;
PFK: Phosphofructokinase;
PGAM: Phosphoglycerate mutase;
PKM2: Pyruvate kinase M2;
LDH: Lactate dehydrogenase;
PDK: Pyruvate dehydrogenase kinase;
G6PD: Glucose-6-phosphate dehydrogenase;
TKTL1: Transketolase-like enzyme 1

According to one embodiment of this aspect of the present invention, the agent is an inhibitor of a glucose transporter.

As used herein, the term “glucose transporter” refers to a protein that transports compounds (whether glucose, glucose analogs, other sugars such as fructose or inositol, or non-sugars such as ascorbic acids) across a cell membrane and are members of the glucose transporter “family” based on structural similarity (e.g., homology to other glucose transport proteins). Glucose transporters also include transporter proteins that have a primary sugar substrate other than glucose. For example, the glucose transporter GLUTS is primarily a transporter of fructose, and is reported to transport glucose itself with low affinity. Similarly, the primary substrate for the glucose transporter HMIT is myo-inositol (a sugar alcohol). Examples of glucose transporter include, but are not limited to GLUT1-12, HMIT and SGLT1-6 transporters.

According to a particular embodiment, the glucose transporter is GLUT-2.

An example of a glucose transporter inhibitor (e.g. a GLUT-2 transporter) is a flavonoid, such as a flavonol (e.g. a quercetin selected from the group consisting of aglycone quercetin, quercetin glycoside, and isoquercetin).

Another example of an inhibitor of a glucose transporter contemplated by the present invention is a cell-permeable thiazolidinedione compound marketed by Calbiochem (catalogue number 400035).

According to a particular embodiment, the agent inhibits a sodium-glucose cotransporter 1 and 2 (SGLT1/2) inhibitors—non-limiting examples being Dapagliflozin is a SGLT2 inhibitor specific to the kidneys; canagliflozin (Invokana, Janssen pharmaceuticals), dapagliflozin (Forxiga [known as Farxiga in the USA]), (Jardiance), and ertugliflozin.

Other contemplated GLUT2 transporter inhibitors include: sappanin-type (SAP) homoisoflavonoids (PMID: 29533635), quercetin, myricetin, isoquercitrin (PMID: 17172639), Phloretin, and Phlorizin (dual inhibitor).

According to a particular embodiment, the agent is selected from the group consisting of Phloretin, Phlorizin and empagliflozin.

According to a particular embodiment, the agent downregulates expression of the glucose transporter.

As used herein the phrase “downregulates expression” refers to downregulating the expression of a protein at the genomic (e.g. homologous recombination and site-specific endonucleases) and/or the transcript level using a variety of molecules which interfere with transcription and/or translation (e.g., RNA silencing agents).

For the same culture conditions the expression is generally expressed in comparison to the expression in a cell of the same species but not contacted with the agent or contacted with a vehicle control, also referred to as control.

Down-regulation of expression may be either transient or permanent.

According to specific embodiments, down regulating expression refers to the absence of mRNA and/or protein, as detected by RT-PCR or Western blot, respectively.

According to other specific embodiments down regulating expression refers to a decrease in the level of mRNA and/or protein, as detected by RT-PCR or Western blot, respectively. The reduction may be by at least a 10%, at least 20%, at least 30%, at least 40%, at least 50%, at least 60%, at least 70%, at least 80%, at least 90%, at least 95% or at least 99% reduction.

Down-regulation at the nucleic acid level is typically effected using a nucleic acid agent, having a nucleic acid backbone, DNA, RNA, mimetics thereof or a combination of same which hybridizes to the endogenous glucose transporter encoding sequence (DNA or RNA, depending on the particular agent) of the cell. The nucleic acid agent may be encoded from a DNA molecule or provided to the cell per se (i.e. the RNA molecule is delivered directly to the cell).

Following is a description of various exemplary methods used to downregulate expression of a gene of interest (e.g. glucose transporter) and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDS) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—

Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—

Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

CRISPR-Cas System—

Many bacteria and archaea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

In order to cut DNA at a specific site, Cas9 proteins require the presence of a gRNA and a protospacer adjacent motif (PAM), which immediately follows the gRNA target sequence in the targeted polynucleotide gene sequence. The PAM is located at the 3′ end of the gRNA target sequence but is not part of the gRNA. Different Cas proteins require a different PAM. Accordingly, selection of a specific polynucleotide gRNA target sequence (e.g., on the glucose transporter nucleic acid sequence) by a gRNA is generally based on the recombinant Cas protein used.

The gRNA comprises a “gRNA guide sequence” or “gRNA target sequence” which corresponds to the target sequence on the target polynucleotide gene sequence that is followed by a PAM sequence.

The gRNA may comprise a “G” at the 5′ end of the polynucleotide sequence. The presence of a “G” in 5′ is preferred when the gRNA is expressed under the control of the U6 promoter. The CRISPR/Cas9 system of the present invention may use gRNA of varying lengths. The gRNA may comprise at least a 10 nts, at least 11 nts, at least a 12 nts, at least a 13 nts, at least a 14 nts, at least a 15 nts, at least a 16 nts, at least a 17 nts, at least a 18 nts, at least a 19 nts, at least a 20 nts, at least a 21 nts, at least a 22 nts, at least a 23 nts, at least a 24 nts, at least a 25 nts, at least a 30 nts, or at least a 35 nts of the target glucose transporter DNA sequence which is followed by a PAM sequence. The “gRNA guide sequence” or “gRNA target sequence” may be at least 17 nucleotides (17, 18, 19, 20, 21, 22, 23), preferably between 17 and 30 nts long, more preferably between 18-22 nucleotides long. In an embodiment, gRNA guide sequence is between 10-40, 10-30, 12-30, 15-30, 18-30, or 10-22 nucleotides long.

Although a perfect match between the gRNA guide sequence and the DNA sequence on the targeted gene is preferred, a mismatch between a gRNA guide sequence and target sequence on the gene sequence of interest is also permitted as along as it still allows hybridization of the gRNA with the complementary strand of the gRNA target polynucleotide sequence on the targeted gene. A seed sequence of between 8-12 consecutive nucleotides in the gRNA, which perfectly matches a corresponding portion of the gRNA target sequence is preferred for proper recognition of the target sequence. The remainder of the guide sequence may comprise one or more mismatches. In general, gRNA activity is inversely correlated with the number of mismatches. Preferably, the gRNA of the present invention comprises 7 mismatches, 6 mismatches, 5 mismatches, 4 mismatches, 3 mismatches, more preferably 2 mismatches, or less, and even more preferably no mismatch, with the corresponding gRNA target gene sequence (less the PAM). Preferably, the gRNA nucleic acid sequence is at least 90%, 91%, 92%, 93%, 94%, 95%, 96%, 97%, 98% and 99% identical to the gRNA target polynucleotide sequence in the gene of interest (e.g., glucose transporter). Of course, the smaller the number of nucleotides in the gRNA guide sequence the smaller the number of mismatches tolerated. The binding affinity is thought to depend on the sum of matching gRNA-DNA combinations.

Any gRNA guide sequence can be selected in the target gene, as long as it allows introducing at the proper location, the patch/donor sequence of the present invention. Accordingly, the gRNA guide sequence or target sequence of the present invention may be in coding or non-coding regions of the glucose transporter gene (i.e., introns or exons).

The number of gRNAs administered to or expressed in a cell (or subject) or subject in accordance with the methods of the present invention may be at least 1 gRNA, at least 2 gRNAs, at least 3 gRNAs at least 4 gRNAs, at least 5 gRNAs, at least 6 gRNAs, at least 7 gRNAs, at least 8 gRNAs, at least 9 gRNAs, at least 10 gRNAs, at least 11 gRNAs, at least 12 gRNAs, at least 13 gRNAs, at least 14 gRNAs, at least 15 gRNAs, at least 16 gRNAs, at least 17 gRNAs, or at least 18 gRNAs. The number of gRNAs administered to or expressed in a cell may be between at least 1 gRNA and at least 15 gRNAs, at least 1 gRNA to and least 10 gRNAs, at least 1 gRNA and at least 8 gRNAs, at least 1 gRNA and at least 6 gRNAs, at least 1 gRNA and at least 4 gRNAs, at least 1 gRNA to and least 3 gRNAs, at least 2 gRNA and at least 5 gRNAs, at least 2 gRNA and at least 3 gRNAs. Different or identical gRNAs may be used to cut the endogenous target gene of interest and liberate the donor/patch nucleic acid, when provided in a vector.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

The Cas protein that may be used in accordance with the present invention has a nuclease (or nickase) activity to introduce a double stranded break (DSB) (or two single stranded breaks (SSBs) in the case of a nickase) in cellular DNA when in the presence of appropriate gRNA(s).

In one embodiment, the Cas9 protein is a recombinant protein.

In another embodiment, the Cas9 protein is derived from a naturally occurring Cas9 which has nuclease activity and which function with the gRNAs of the present invention to introduce double stranded breaks in the targeted DNA.

In an embodiment, the Cas9 protein is a dCas9 protein (i.e., a mutated Cas9 protein devoid of nuclease activity) fused with a dimerization-dependent Fokl nuclease domain. In another embodiment, the Cas protein is a Cas9 protein having a nickase activity.

Cas9 proteins are natural effector proteins produced by numerous species of bacteria including Streptococcus pyogene, Streptococcus thermophiles, Staphylococcus aureus, and Neisseria meningitides. Accordingly, in an embodiment, the Cas protein of the present invention is a Cas9 nuclease/nickase derived from Streptococcus pyogene, Streptococcus thermophiles, Staphylococcus aureus or Neisseria meningitides. In an embodiment, the Cas9 recombinant protein of the present invention is a human-codon optimized Cas9 derived from S. pyogenes (hSpCas9). In an embodiment, the Cas9 recombinant protein of the present invention is a human-codon optimized Cas9 derived from S. aureus (hSaCas9).

Non-limiting examples of viral vectors which can be used to express the Cas9 and/or gRNA include retrovirus, lentivirus, Herpes virus, adenovirus or adeno Associated Virus, as well known in the art. Herpesvirus, adenovirus, Adeno-Associated virus and lentivirus derived viral vectors have been shown to efficiently infect neuronal cells. Preferably, the viral vector is episomal and not cytotoxic to cells. In an embodiment, the viral vector is an AAV or a Herpes virus.

Downregulation of a glucose transporter can be also achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

In one embodiment, the RNA silencing agents of the present invention (including gRNAs which are further described herein above) are modified polynucleotides. Polynucleotides can be modified using various methods known in the art.

For example, the oligonucleotides or polynucleotides of the present invention may comprise heterocylic nucleosides consisting of purines and the pyrimidines bases, bonded in a 3′-to-5′ phosphodiester linkage.

Preferably used oligonucleotides or polynucleotides are those modified either in backbone, internucleoside linkages, or bases, as is broadly described hereinunder.

Specific examples of preferred oligonucleotides or polynucleotides useful according to this aspect of the present invention include oligonucleotides or polynucleotides containing modified backbones or non-natural internucleoside linkages. Oligonucleotides or polynucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide or polynucleotide backbones include, for example: phosphorothioates; chiral phosphorothioates; phosphorodithioates; phosphotriesters; aminoalkyl phosphotriesters; methyl and other alkyl phosphonates, including 3′-alkylene phosphonates and chiral phosphonates; phosphinates; phosphoramidates, including 3′-amino phosphoramidate and aminoalkylphosphoramidates; thionophosphoramidates; thionoalkylphosphonates; thionoalkylphosphotriesters; and boranophosphates having normal 3′-5′ linkages, 2′-5′ linked analogues of these, and those having inverted polarity wherein the adjacent pairs of nucleoside units are linked 3′-5′ to 5′-3′ or 2′-5′ to 5′-2′. Various salts, mixed salts, and free acid forms of the above modifications can also be used.

Alternatively, modified oligonucleotide or polynucleotide backbones that do not include a phosphorus atom therein have backbones that are formed by short-chain alkyl or cycloalkyl internucleoside linkages, mixed heteroatom and alkyl or cycloalkyl internucleoside linkages, or one or more short-chain heteroatomic or heterocyclic internucleoside linkages. These include those having morpholino linkages (formed in part from the sugar portion of a nucleoside); siloxane backbones; sulfide, sulfoxide, and sulfone backbones; formacetyl and thioformacetyl backbones; methylene formacetyl and thioformacetyl backbones; alkene-containing backbones; sulfamate backbones; methyleneimino and methylenehydrazino backbones; sulfonate and sulfonamide backbones; amide backbones; and others having mixed N, O, S and CH2 component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides or polynucleotides which may be used according to the present invention are those modified in both sugar and the internucleoside linkage, i.e., the backbone of the nucleotide units is replaced with novel groups. The base units are maintained for complementation with the appropriate polynucleotide target. An example of such an oligonucleotide mimetic includes a peptide nucleic acid (PNA). A PNA oligonucleotide refers to an oligonucleotide where the sugar-backbone is replaced with an amide-containing backbone, in particular an aminoethylglycine backbone. The bases are retained and are bound directly or indirectly to aza-nitrogen atoms of the amide portion of the backbone. United States patents that teach the preparation of PNA compounds include, but are not limited to, U.S. Pat. Nos. 5,539,082; 5,714,331; and 5,719,262; each of which is herein incorporated by reference. Other backbone modifications which may be used in the present invention are disclosed in U.S. Pat. No. 6,303,374.

Additionally, or alternatively the oligonucleotides/polynucleotide agents of the present invention may be phosphorothioated, 2-o-methyl protected and/or LNA modified.

Oligonucleotides or polynucleotides of the present invention may also include base modifications or substitutions. As used herein, “unmodified” or “natural” bases include the purine bases adenine (A) and guanine (G) and the pyrimidine bases thymine (T), cytosine (C), and uracil (U). “Modified” bases include but are not limited to other synthetic and natural bases, such as: 5-methylcytosine (5-me-C); 5-hydroxymethyl cytosine; xanthine; hypoxanthine; 2-aminoadenine; 6-methyl and other alkyl derivatives of adenine and guanine; 2-propyl and other alkyl derivatives of adenine and guanine; 2-thiouracil, 2-thiothymine, and 2-thiocytosine; 5-halouracil and cytosine; 5-propynyl uracil and cytosine; 6-azo uracil, cytosine, and thymine; 5-uracil (pseudouracil); 4-thiouracil; 8-halo, 8-amino, 8-thiol, 8-thioalkyl, 8-hydroxyl, and other 8-substituted adenines and guanines; 5-halo, particularly 5-bromo, 5-trifluoromethyl, and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Additional modified bases include those disclosed in: U.S. Pat. No. 3,687,808; Kroschwitz, J. I., ed. (1990), “The Concise Encyclopedia Of Polymer Science And Engineering,” pages 858-859, John Wiley & Sons; Englisch et al. (1991), “Angewandte Chemie,” International Edition, 30, 613; and Sanghvi, Y. S., “Antisense Research and Applications,” Chapter 15, pages 289-302, S. T. Crooke and B. Lebleu, eds., CRC Press, 1993. Such modified bases are particularly useful for increasing the binding affinity of the oligomeric compounds of the invention. These include 5-substituted pyrimidines, 6-azapyrimidines, and N-2, N-6, and O-6-substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S. et al. (1993), “Antisense Research and Applications,” pages 276-278, CRC Press, Boca Raton), and are presently preferred base substitutions, even more particularly when combined with 2′-O-methoxyethyl sugar modifications.

The modified polynucleotide of the present invention may also be partially 2′-oxymethylated, or more preferably, is fully 2′-oxymethylated.

The RNA silencing agents (including gRNAs) designed according to the teachings of the present invention can be generated according to any oligonucleotide synthesis method known in the art, including both enzymatic syntheses or solid-phase syntheses. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

According to an embodiment of the invention, the RNA silencing agent (including the gRNA described herein) is specific to the target RNA (e.g., glucose transporter) and does not cross inhibit or silence a gene or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplates use of dsRNA to downregulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

In particular, the invention according to some embodiments thereof contemplates introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433. and Diallo et al, Oligonucleotides, Oct. 1, 2003, 13(5): 381-392. doi: 10.1089/154545703322617069.

The invention according to some embodiments thereof also contemplates introduction of long dsRNA specifically designed not to induce the interferon and PKR pathways for downregulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base-pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of some embodiments of the invention may also be a short hairpin RNA (shRNA).

According to another embodiment the RNA silencing agent may be a miRNA or a miRNA mimic.

The term “microRNA mimic” refers to synthetic non-coding RNAs that are capable of entering the RNAi pathway and regulating gene expression. miRNA mimics imitate the function of endogenous microRNAs (miRNAs) and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

It will be appreciated from the description provided herein above, that administration of a miRNA may be affected in a number of ways:
1. Administering the mature double stranded miRNA;
2. Administering an expression vector which encodes the mature miRNA;
3. Administering an expression vector which encodes the pre-miRNA. The pre-miRNA sequence may comprise from 45-90, 60-80 or 60-70 nucleotides. The sequence of the pre-miRNA may comprise a miRNA and a miRNA* as set forth herein. The sequence of the pre-miRNA may also be that of a pri-miRNA excluding from 0-160 nucleotides from the 5′ and 3′ ends of the pri-miRNA.
4. Administering an expression vector which encodes the pri-miRNA The pri-miRNA sequence may comprise from 45-30,000, 50-25,000, 100-20,000, 1,000-1,500 or 80-100 nucleotides. The sequence of the pri-miRNA may comprise a pre-miRNA, miRNA and miRNA*, as set forth herein, and variants thereof.

Another agent capable of downregulating a polypeptide (e.g. glucose transporter) is a DNAzyme molecule capable of specifically cleaving an mRNA transcript or DNA sequence of the caspase. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Downregulation of a polypeptide (e.g. glucose transporter) can also be effected by using an antisense polynucleotide capable of specifically hybridizing with an mRNA transcript encoding the glucose transporter.

Design of antisense molecules which can be used to efficiently downregulate a glucose transporter must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

Another agent capable of downregulating a polypeptide (e.g. glucose transporter) is a ribozyme molecule capable of specifically cleaving an mRNA transcript encoding the glucose transporter. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications.

An additional method of regulating the expression of a polypeptide (e.g. glucose transporter) gene in cells is via triplex forming oligonucleotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al, Science, 1992; 251:1360-1363; Cooney, M., et al., Science, 1988; 241:456-459; and Hogan, M. E., et al., EP Publication 375408. Modification of the oligonuclotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch, BMC Biochem, 2002 Sep. 12, Epub). The same authors have demonstrated that TFOs designed according to the A-AT and G-GC rule do not form non-specific triplexes, indicating that the triplex formation is indeed sequence specific.

Thus for any given sequence in the glucose transporter regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-1 gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Agents that block lipid synthesis are also contemplated. In general any lipid synthesis blocking drug that bypasses the liver will block lipid synthesis mainly in the kidney. This can be achieved by conjugating lipid synthesis blockers to moieties that specifically target the kidney, by using blockers that are highly hydrophobic (bypassing the liver) or compounds which are not CYP450 substrates. A non-limiting example of such an agents is BMS-303141 is an ACLY inhibitor.

Other contemplated agents include those that upregulate lipid oxidation in the kidney. Agents that work via this mechanism include agonists of PPARA, preferably fibrate drugs, a class of amphipathic carboxylic acids (clofibrate, gemfibrozil, ciprofibrate, bezafibrate, and fenofibrate). Dual PPARA/G agonists including saroglitazar, aleglitazar, muraglitazar and tesaglitazar. Also CP 775146, GW 7647, Oleylethanolamide, Palmitoylethanolamide, WY 14643 are also contemplated.

The agents that protect the polarity of proximal tubule epithelial cells from the toxic effect of said kidney damaging agent (e.g. those that decrease lipid accumulation) may be administered to the subject per se or as part of a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent that reduces lipid accumulation in the kidney accountable for the therapeutic effect in the kidney.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the CNS include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of an aggregate of cells having a similar structure and/or a common function. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (agent that decreases lipid accumulation) effective to prevent, alleviate or ameliorate symptoms of a disorder (e.g., cancer/anthrax infection) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue or blood levels of the active ingredient which are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

In the context of a combination therapy, the kidney-damaging agent may be administered by the same route of administration (e.g. intrapulmonary, oral, enteral, etc.) as the agent that protects the polarity of proximal tubule epithelial cells from the toxic effect of said kidney damaging agent (e.g. the inhibitor of glucose reabsorption) is administered. In the alternative, the kidney-damaging agent may be administered by a different route of administration to the protective agent (e.g. the inhibitor of glucose reabsorption).

The kidney-damaging agent can be administered immediately prior to (or after) the protective agent, on the same day as, one day before (or after), one week before (or after), one month before (or after), or two months before (or after) the protective agent, and the like.

The kidney-damaging agent and the protective agent (e.g. the inhibitor of glucose reabsorption) can be administered concomitantly, that is, where the administering for each of these reagents can occur at time intervals that partially or fully overlap each other. They may be administered in a single formulation or in separate formulations. The kidney-damaging agent and the protective agent can be administered during time intervals that do not overlap each other. For example, the kidney-damaging agent can be administered within the time frame of t=0 to 1 hours, while the protective agent can be administered within the time frame of t=1 to 2 hours. Also, the kidney-damaging agent can be administered within the time frame of t=0 to 1 hours, while the protective agent can be administered somewhere within the time frame of t=2-3 hours, t=3-4 hours, t=4-5 hours, t=5-6 hours, t=6-7 hours, t=7-8 hours, t=8-9 hours, t=9-10 hours, and the like. Moreover, the protective can be administered somewhere in the time frame of t=minus 2-3 hours, t=minus 3-4 hours, t=minus 4-5 hours, t=5-6 minus hours, t=minus 6-7 hours, t=minus 7-8 hours, t=minus 8-9 hours, t=minus 9-10 hours.

It will be appreciated that the protective agent may be formulated in a single composition together with the nephrotoxic agent.

Thus, the present invention contemplates compositions (e.g. pharmaceutical compositions) comprising a single acceptable carrier and, as active agents:

(i) a kidney-damaging therapeutic agent; and

(ii) an agent that protects the polarity of proximal tubule epithelial cells from the toxic effect of said kidney damaging agent (e.g. the inhibitor of glucose reabsorption).

The protective agent of the present invention and the kidney-damaging agent are typically provided in combined amounts to achieve therapeutic and/or prophylactic effectiveness. This amount will evidently depend upon the particular compound selected for use, the nature and number of the other treatment modality, the condition(s) to be treated, prevented and/or palliated, the species, age, sex, weight, health and prognosis of the subject, the mode of administration, effectiveness of targeting, residence time, mode of clearance, type and severity of side effects of the composition and upon many other factors which will be evident to those of skill in the art. The kidney-damaging agent will be used at a level at which a therapeutic or prophylactic effect in combination with the protective agent is observed.

The kidney-damaging agent may be administered (together with the protective agent) at a gold standard dosing as a single agent, below a gold standard dosing as a single agent or above a gold standard dosing as a single agent.

According to specific embodiments, the kidney-damaging agent is administered above the gold standard dosing as a single agent.

As used herein the term “gold standard dosing” refers to the dosing which is recommended by a regulatory agency (e.g., FDA), for a given tumor at a given stage.

According to specific embodiments, the kidney-damaging agent is administered using a regimen which is different to the gold standard regimen when used as a single agent (e.g. it may be provided for a longer length of time).

According to other specific embodiments, the kidney-damaging agent is administered (in combination with the protective agent) at a dose that is associated with kidney damage when used as a single agent.

Thus, in one embodiment, the amount of the kidney-damaging agent (when used in combination therapy) is above the minimum dose used for therapeutic or prophylactic effectiveness when used as a single therapy (e.g. 110%, or 125% to 175% of the minimum dose). The therapy is rendered more effective because higher doses of the active agent can be used to treat the disease whilst the protective agent decreases the negative side-effect of renal toxicity, the combinations are effective overall.

In an alternative embodiment, the kidney-damaging agent of the present invention and the protective agent are synergistic with respect to their side effects. That is to say that the side-effects caused by the protective agent in combination with the kidney-damaging agent are less than would be anticipated when the equivalent therapeutic effect is provided by either the kidney-damaging agent when used separately.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

It is expected that during the life of a patent maturing from this application many relevant useful agent that have the negative side-effect of being kidney damaging will be developed and the scope of the term kidney damaging agent is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Organ-On-Chip Analysis of Kidney Metabolism Reveals Glucose-Driven Lipotoxicity as a Mechanism of Drug-Induced Nephrotoxicity

Materials and Methods

Cell Culture

All cells were cultured under standard conditions in a humidified incubator at 37° C., under 5% CO₂. HK-2 cells were cultured in Dulbecco's Modified Eagle Medium/Nutrient Mixture F-12 basal medium (DMEM/F-12, Sigma, USA) supplemented with 10% fetal bovine serum (BI, Israel), 5 ng ml⁻¹ Epithelial Growth Factor (EGF, Peprotech, USA), L-Alanyl-L-Glutamine (BI, Israel), 100 U ml⁻¹ penicillin, and 100 μg ml⁻¹ streptomycin (BI, Israel).

Renal Proximal Tubule Epithelial Cells (RPTEC) were purchased from Lonza (Basel, Switzerland). Cells were cultured in MCDB 153 basal medium (Sigma-Aldrich, USA) supplemented with 0.5% fetal bovine serum (BI, Israel), Insulin, transferrin and selenium (ITS, Gibco, USA), 0.1 μM dexamethasone (Sigma-Aldrich, USA), 10 ng ml⁻¹ Epithelial Growth Factor (EGF, Peprotech, USA), 5 pM Triiodothyronine (T3, Sigma, USA), 0.5 μg ml⁻¹ Epinephrin (Sigma, USA), 100 U ml⁻¹ penicillin, and 100 μg ml⁻¹ streptomycin (BI, Israel). Primary cells were cultured from passage 0 to 3, up to 8 population doublings.

Primary rat cardiac microvascular endothelial cells (RCEC, Vec Technologies®, USA) were cultured on gelatin-coated flasks with Endothelial Cell Basal medium-2 (EBM™-2, Lonza, Switzerland) supplemented with EGM™-2 BulletKit™ (Lonza, Switzerland).

Quantitative RT-PCR

RNA was isolated and purified using RNeasy® Mini kit (Qiagen, Germany) according to manufacturer instructions. RNA concentration and purity were determined using NanoDrop ND-1000 spectrophotometer (Thermo Fisher Scientific). cDNA was synthesized from 1 μg RNA sample using qScript cDNA SuperMix (Quanta BioSciences, USA) according to the manufacturer's protocol. Gene expression analysis was carried out using KAPA SYBR FAST Universal 2×qPCR Master Mix (Kapa Biosystems, USA) on Applied Biosystems QuantStudio 5 System for 384-well plate format (Applied Biosystens, USA), according to manufacturer's directions. Gene transcription was evaluated using the ΔΔCt method normalized to 60S ribosomal protein L32 (RPL32) or ubiquitin C (UBC).

3D Cyst Formation

Matrigel® basement membrane matrix (cat: 356231, Corning®, Carlsbad, Calif.) was thawed for 2-12 hours on ice under cold conditions (<4° C.). The bottom of each well of a black 96-well plate glass bottom (Greiner Bio-one, Austria) was coated with 30 μL gel solution, while avoiding bubble formation. The plate was incubated for 30 minutes at 37° C. The same procedure was repeated with 45 μL of gel solution for each 8-well cover-glass slide (Nunc™ Lab-Tek™ II Carlsbad, Calif.) for higher resolution confocal microscopy.

HK-2 or RPTEC cells were suspended in an ice-cold gel solution at a density of 15×10⁴ cells/mL. 70 μl of the gel-cell mixture was added to each pre-coated well and incubated for 60 minutes at 37° C. and 5% CO₂. The final cell concentration was 10-500 cells per well. HK-2 or RPTEC culture medium was added and the plates were incubate at 37° C., 5% CO₂ for 2 weeks for HK-2s and up to 4 weeks for RPTECs, with daily media changes, until mature cysts with a single central lumen were visible. At first, tightly adherent spheroids formed and later on a single central lumen was generated, via membrane separation and apoptosis.

Functional Polarity Assay

The medium was removed from the top of the gel-containing cysts, and replaced with fresh culture medium comprising 2 μM Calcein AM (Molecular Probes). Following a one hour incubation at 37° C., 5% CO₂, the medium was removed and replaced with fresh culture medium prior to imaging. Polarized cysts will expel Calcein AM through their apical membrane to the lumen, and therefore polarized cysts will show only very low green intensity under the fluorescent microscope. If the polarity is disturbed, cells will retain Calcein AM in their cytoplasm and show high green intensity when excited with the same intensity.

Bioreactor Design and Fabrication

Bioreactor manifold and disposable polydimethylsiloxane (PDMS) microwell insert design was carried out using AutoCAD® (Autodesk, USA), and adapted for computer numerical control (CNC) using SolidWorks® (SolidWorks, USA). Bioreactor manifold was machined from biocompatible polyetherimide (ULTEM) blocks using Haas VF-2SSYT (Hass Automations, USA) machining. Each unit was composed of two 50.8-mm circular support structures that fit standard 2-inch inserts, imbedded with a biocompatible epoxy-glued glass window for efficient light transmission and a stainless-steel needle connection for perfusion.

PDMS microwell inserts were fabricated by laser cutting. Briefly, a thin sheet of PDMS (Dow Corning) was cast to 0.7 mm height using a motorized film applicator (Erichsen) and cured at 70° C. for 1 h. Microwells were cut to 1.5 mm diameter, and a center-to-center distance of 3 mm using a 355-nm pulsed Nd-YAG laser (3D-Micromac). PDMS inserts were washed with 70% (vol/vol) EtOH, nitrogen dried, and covalently bound to clean 0.5-mm thick glass coverslips (Schott) using oxygen plasma activation.

Sealing around the microwells was carried out with a rubber gasket creating a perfusion chamber with an internal volume of 200 μL. Pressure and sealing was maintained using 4 stainless steel screws. Each bioreactor was perfused separately via 0.03″ Tygon® low adhesion tubing (Saint-Gobain, France) fitted onto the steel needles.

Organoid Seeding

Polydimethylsiloxane (PDMS) microwell inserts were sterilized with 70% EtOH and 30-min exposure to UV light prior to cell seeding. Kidney cells were trypsinized, counted, and centrifuged at 300×g for 5 min at 4° C. The pellet was then mixed with 400 μg of 50-micrometer-diameter polystyrene microbeads were loaded with ruthenium-phenanthroline-based phosphorescence dye (CPOx-50-RuP oxygen-sensing microbeads; Colibri Photonics, Germany) and re-suspended in 100 μl of ice-cold solution of Matrigel® basement membrane matrix (cat: 356231, Corning®, Carlsbad, Calif.) for a final seeding density of 7.5×10⁴ cells/well. Then, 1.35 μl of Matrigel® suspension containing cells and oxygen-sensing beads was seeded in each well using PIPETMAN M P10M (Gilson, UK). The inoculated microwell insert was then incubated for 30 min at 37° C. to polymerize the gel. Following polymerization, the insert was immersed in 3 ml of cell culture medium and incubated overnight at 37° C. prior to being sealed in the bioreactor housing. Bioreactors were then placed in a climate control chamber on an IX81 fluorescence microscope (Olympus, Japan). Bioreactors were continuously perfused with cell culture medium noted above supplemented with 10 mM HEPES and 1% DMSO at a flow rate of 5 μL/min. Drug induction started upon metabolic stabilization. Stabilization was assessed by daily measurements of oxygen and metabolites. Stabilization occurred usually after 3-4 days.

Real-Time Oxygen Measurement

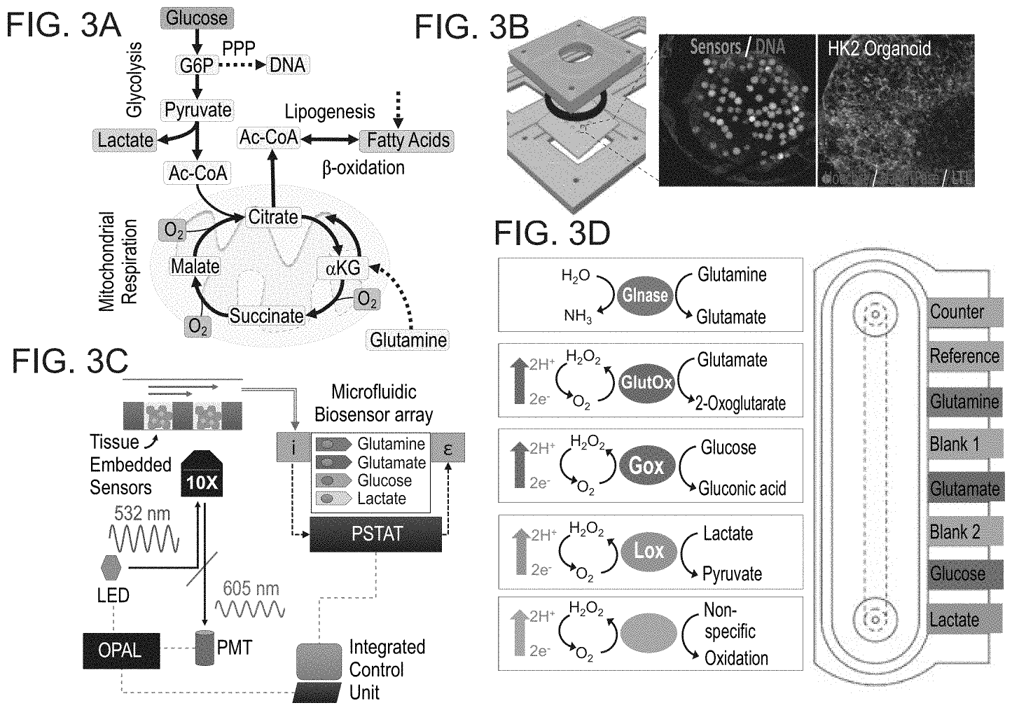

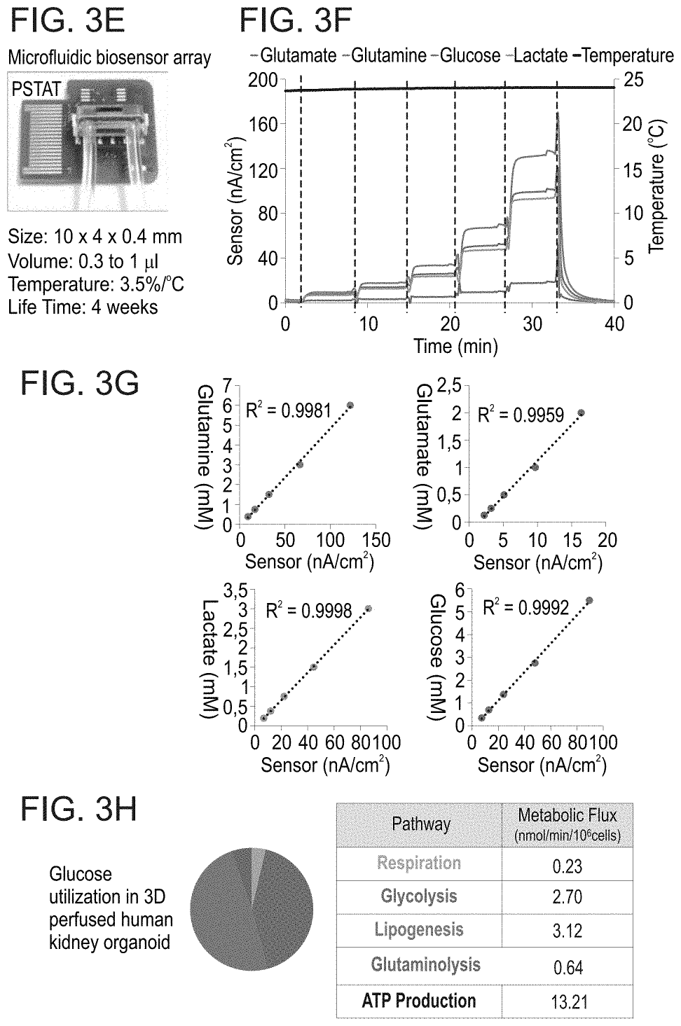

Real-time oxygen measurements were performed optically using on-chip lifetime-based luminescence quenching (LBLQ). RuP phosphorescence signal shows a characteristic delay given by the lifetime of its excited triplet state. Oxygen acts as a quencher, leading to a decrease in decay time and signal intensity with increasing concentration. We chose to measure decay time, rather than signal intensity, as it is not sensitive to changes in probe concentration or excitation intensity over the course of the experiment. The signal was measured using the OPAL system (Colibri Photonics, Germany) that comprises of a control module, 532 nm LED excitation source, and a photomultiplier (PMT) detector mounted on the ocular of an IX81 Olympus microscope (Olympus, Japan). A filter cube with 531/40 (Ex), 555, 607/70 (Em) was inserted in the optical light path during measurements (FIG. 3C). To accurately measure decay time, we chose phase modulation in which sinusoidal amplitude-modulated light is shifted in phase due to oxygen quenching. To overcome the superposition of in-phase background fluorescence that alters the phase of the detected signal, a novel 53.5 and 31.3 kHz two-frequency phase modulation was used that allowed for the screening out of interference. Measurements were carried out by averaging five consecutive 4-s exposures. Measurements were taken every 15 min. Under similar conditions, 28 days of measurement of organoids was carried out with no apparent phototoxicity, signal drift, or relevant loss of signal intensity.

Assessment of Cellular Toxicity and Time to Onset

Bioreactors were perfused with different concentrations of compounds dissolved in culture medium. Cell viability was determined by oxygen uptake following 24 hours of exposure unless otherwise noted. TC₅₀ concentrations were determined using MATLAB by sigmoidal curve fitting. All error bars indicate ±95% Confidence range. Time to onset was analyzed by MATLAB based on LPF and trend assessment.

Real-Time Glucose and Lactate Measurements

Amperometric glucose and lactate sensors were purchased from Innovative Sensor Technology (IST, Switzerland). The sensors are based on the enzymatic reactions of glucose oxidase, with a linear range of 0.5 mM to 30 mM, and lactate oxidase, with a linear range of 0.5 mM to 20 mM. Both sensors produce H₂O₂ in amounts proportional to the measured metabolite, which is detected with platinum electrodes under polarized condition. Measurements were made continuously over the whole experiment, 8-24 hours prior to exposure and until respiratory response was confirmed. Measurements were carried out and calibrated to sensitivity decrease by on-chip potentiostat (IST, Switzerland).

Metabolic Pathways and ATP Production

Glucose uptake, oxygen uptake, and lactate production rates were measured by calculating the change in metabolite concentration between the bioreactor in- and outflow as a function of perfusion rate and cell number. Metabolic rates were calculated assuming negligible contribution to oxygen uptake by fatty acid oxidation and enzymatic activity. Low level of lipids in the culture medium ensured that fatty acid uptake was more than 50-fold lower than glucose, whereas glutamine contribution to the Krebs cycle was minor and glycogen content following 12-hour exposure to the drug showed no significant change.

Based on these assumptions, oxidative phosphorylation flux was calculated by dividing the oxygen uptake rate by six. It was estimated that 32 ATP molecules were generated by complete oxidation of one molecule of glucose. Glycolysis flux was calculated by dividing lactate production rate by two, with maximal rate defined by glucose uptake rate minus the oxidative phosphorylation flux. ATP production in glycolysis was estimated to be two molecules per molecule of glucose. It was assumed that any glucose left over was directed toward lipogenesis, because the contribution of pentose phosphate pathway in non-proliferating cells is minor. Finally, it was assumed that excess lactate was produced by glutaminolysis, and assumption proved by off-chip measurement of glutamine uptake. ATP production in glutaminolysis was estimated to be three molecules per molecule of lactate generated.

Glucose Accumulation Assay

To monitor glucose uptake in cells, a fluorescent analog of glucose 2-(N-(7-Nitrobenz-2-oxa-1,3-diazol-4-yl) Amino)-2-Deoxyglucose (2-NDBG), (Invitrogen, CA, USA) was used. After overnight drug exposure, the medium was removed and replaced with Dulbecco's Phosphate Buffered Saline (Sigma, USA) with 17 mM and 6 mM of 2-NDBG to match glucose concentration of HK-2 and RPTEC culture medium respectively, incubated for 1 hour at 37° C., 5% CO₂ before imaging. 2-NDBG is taken up in mammalian cells by GLUT-2, if its activity or shuttling is disturbed, 2-NDBG will accumulate in the cells, showing higher green intensity.

Lipid Accumulation Assay

Quantification of lipid accumulation was performed using HCS LipidTOX™ Phospholipidosis and Steatosis Detection Kit (ThermoFisher). Briefly, HK-2 and RPTEC cells were incubated with different concentrations of compounds dissolved in culture medium and 1× Phospholipidosis Detection Reagent for 48 h, subsequently fixed in 4% PFA. Cells were then stained with 1× Green LipidTOX™ for neutral lipids for 45 min and counterstained with 1 μg/mL Hoechst 33258. Staining intensity was normalized to number of Hoechst 33258 positive nuclei.

Reduction of Toxicity Experiment

Empagliflozin (5 μM) a potent SGLT2 inhibitor, phloretin (1 mM) inhibits the apicobasal shuttling of the GLUT2, phlorizin (200 μM) is a mild inhibitor of SGLT1 and SGLT2. When all three inhibitors were used together, the mix was referred to as a cocktail. All experiments were performed on a 96 well plate, in triplicates with controls with no drugs, or inhibitors alone. First, cells were incubated for 1 hour with inhibitors alone and then drugs were added with inhibitors for 24 hours for LIVE DEAD assay or 48 hours for Lipid accumulation (LipidTox Assay). Cell viability was determined using LIVE/DEAD Cytotoxicity kit (Molecular Probes, USA) according to manufacturer directions. Briefly, cultures were incubated for 30 min with 2 μM calcein AM and 4 μM ethidium homodimer-1. Live cells were positive for green fluorescence due to hydrolysis of the acetoxymethyl ester group by intracellular esterases. Dead cells were positive for red fluorescence due to ethidium homodimer-1 binding intracellular DNA, possible in intact membranes. Cellular viability was expressed as live over dead ratio and normalized based on negative (DMSO/DDW/PBS) controls. Fluorescence micrographs were analyzed for total fluorescence using ImageJ for 9 repeats for each sample. Error bars indicate ±SEM.

After staining, plates were mounted on the fluorescent microscope for quantification. Rescue was achieved when significant percentage of live cells and/or a reduced amounts of dead cells was observed as compared with cells treated with the drug only. Both viability and lipid accumulation were significantly closer to controls with no drugs when incubated with inhibitors, showing moderated toxicity over time, countering deleterious adverse effects of the drugs.

Statistical Analysis

Experiments were repeated 2 or 3 times with triplicate samples for each experimental condition, unless stated otherwise. Data from representative experiments are presented, and similar trends were seen in multiple trials. A parametric two-tailed Student's t-test was used for calculating significant differences between groups. All error bars represent ±SE, unless otherwise noted

Results

HK2 Cells to Model Human Proximal Tubules, Shows Early Signs of Acute Injury after Sub-Toxic Drug Exposure.

HK-2 (human kidney 2) is a proximal tubular cell (PTC) line derived from normal kidney. The cells were immortalized by transduction with human papilloma virus 16 (HPV-16) E6/E7 genes (FIG. 1A). They express several major transporters for proximal tubule physiology such as P-glycoprotein (MDR1) or the sodium phosphate cotransporter (NaPi2a). HK-2 cells retain functional characteristics of proximal tubular epithelium such as Na+-dependent/phlorizin-sensitive sugar transport and adenylate cyclase responsiveness to parathyroid, but not to antidiuretic hormone. The cells are capable of gluconeogenesis as evidenced by their ability to make and store glycogen. HK-2 cells are anchorage dependent (FIG. 1B). Thus, HK-2 cells can reproduce experimental results obtained with freshly isolated PTCs.

Standard 2D LIVE/DEAD assays of three known nephrotoxic compounds with different mechanisms of action provides TC50 values of 51.3 μM for Cyclosporine A (CsA), 95.1 μM for Cisplatin and 182.4 mM for Gentamicin (FIG. 1C). When exposed to the same drugs at sub-toxic levels, HK-2 cells showed elevated expression of the renal acute injury marker KIM-1. Moreover, KIM-1 immunostainings show expression which is restricted to the perinuclear in controls (i.e. prior to culture with the nephrotoxic compounds) and which becomes membranal following a 24 hour exposure, showing evidence of early signs of damage (FIG. 1D-E). HSP60 immunostainings suggest that at these low concentrations, the mitochondrial network is disturbed. In addition, expression of HSP60 is increased with increasing concentrations of the nephrotoxic compound, even when the concentration of the compounds is below the threshold of cellular damage (FIG. 1D-E).

Sub-Toxic Drug Exposure Disturbs Function in Polarized Three-Dimensional Cysts.

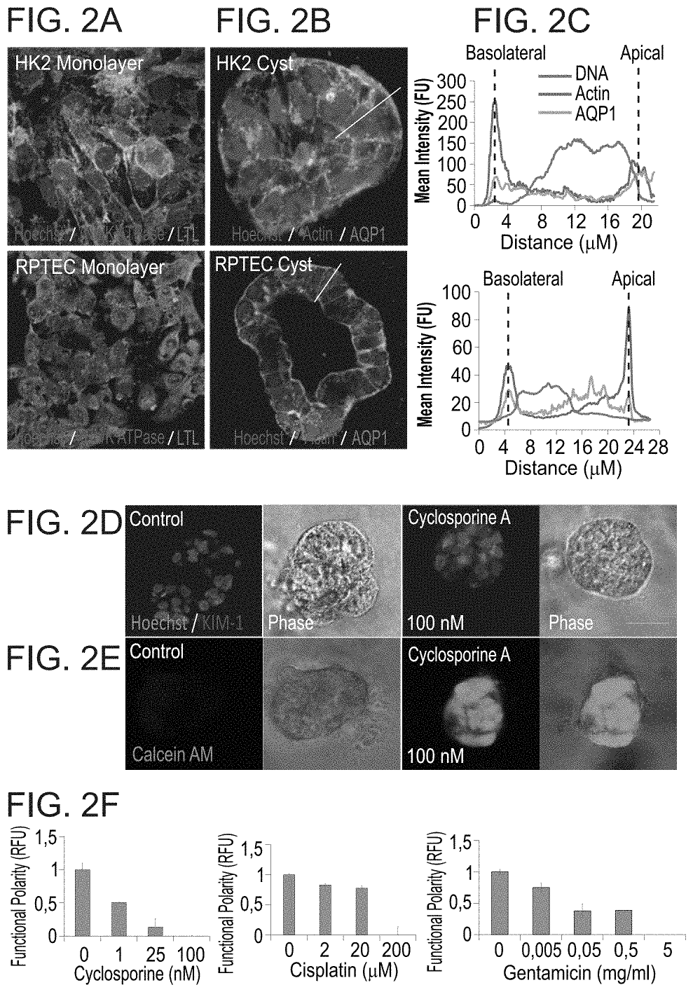

The surface membrane of proximal tubular cells is organized into distinct apical and basolateral membrane domains. 2D monolayers of both HK-2 and RPTEC are positive for proximal tubule apical marker Lotus Tetragonobulus Lectin (LTL) and the basolateral Sodium/Potassium pump (Na/K ATPase), indicating that the cells maintain some polarity after isolation and transformation (FIG. 2A). Furthermore, HK-2 cells are capable of forming three-dimensional cysts in a matrix similar to primary PTCs (RPTEC) that recapitulate the proximal tubule's polarization (FIG. 2B). This translates into formation of 3D cysts with a single lumen when cells are cultured in a gel. The apical membranes facing the lumen show stronger expression of F-actin as shown in the expression profiles (FIG. 2B-C). These organized structures can be used for functional assays modeling the configuration of proximal tubules. They show the same phenomena of KIM-1 membranal expression after sub-toxic drug exposure (FIG. 2D). Alterations in the surface membrane polarity of proximal tubular is a basic indication for malfunction (Molitoris & Wagner, 1996). Calcein AM is a dye uptaken by live cells and cleaved by esterases activity, creating a fluorescent dye. Polarized cells express P-glycoprotein transporter on their apical membranes, also called Multi-drug resistance transporter 1 (MDR1), that effluxes drugs and compounds such as Calcein AM. Thus, functional polarized cysts should eject the dye out into the lumen. Polarized cysts (Control) display low levels of green fluorescence after washing, suggesting that Calcein AM has been exported out of the cells. However, cysts exposed with sub-toxic levels of CsA (100 nM) retained high green fluorescence, indicating that Calcein AM could not be exported from the cells (FIGS. 2E-F). Similar results were found with cisplatin and gentamicin (FIG. 2F). This suggests renal function has been disturbed as indicated by a rapid loss of functional polarity.

Real-Time Measurements of Oxygen and Metabolites of Vascularized Organoids in a Microphysiological Flux Balance Platform.

Central carbon metabolism and fuel utilization are sensitive markers of physiological stress. Flux balance analysis is a computational method to derive intracellular fluxes of central carbon metabolism and fuel utilization by measuring changes in extracellular fluxes. Interestingly, for non-proliferating cells growing in lipid and glutamine poor medium, central carbon fluxes can be estimated by measuring glucose, lactate, glutamine and oxygen fluxes alone (FIG. 3A). To monitor the dynamic transition between these metabolic pathways, a microfluidic system was designed that maintains renal organoids under physiological conditions mimicking proximal tubular physiology while dynamically measuring oxygen, glucose, lactate and glutamine concentrations (FIG. 3B-C). 6-unit bioreactor platform manifold was fabricated from biocompatible polyetherimide (ULTEM™) using CNC, while disposable multi-well microchips were fabricated using laser cutting (FIG. 3B). Oxygen was measured using tissue-embedded microsensors loaded with a ruthenium-based dye, whose phosphorescence is quenched in the presence of oxygen leading to decreasing decay time. In contrast to intensity measurement, decay time is insensitive to probe concentration or excitation intensity. A sinusoidal intensity-modulated light was used resulting in an oxygen-dependent phase shift in the 605-nm emission that is stable down to three particles, and 1.5 mm away from the focus permitting accurate measurement even during toxic damage and subsequent tissue disintegration (FIG. 3C).

A microfluidic biosensor array was integrated into the platform with an on-chip temperature sensor, and a three-electrode design in which the counter and reference electrodes are separated. The reference electrode is used to measure the working electrode potential without passing current through it, while the counter electrode closes a circuit, allowing current to pass. This circuit is not possible in a two-electrode system. Anodic oxidation of H₂O₂ on platinum produces a current rapidly (t₉₀<25 sec), while embedded catalase activity prevents cross-contamination. A 450-mV potential between the working and counter electrodes is monitored against a reference electrode to minimize background noise caused by reversible electrolysis events (FIG. 3D). Finally, an on-chip potentiostat (PSTAT) monitoring an 8-electrode array is integrated in the 10×4×0.4 mm microchip with a total volume of 0.3 to 1 μL suitable to be connected directly to the bioreactor outflow (FIG. 3E). A single central processing unit (CPU) controls the entire system, simplifying synchronization (FIG. 3C-E). Sensors shows linear range from 0.05 mM to 15 mM lactate and 25 mM glucose (FIG. 3F-G). Using our microphysiological platform we calculated the intracellular metabolic fluxes of polarized HK-2 organoids under steady state condition. Glucose utilization in each pathway is shown as nmol/min/10⁶ cells as well as calculated ATP production. Relative glucose utilization is shown as pie chart (FIG. 3H).

Three-Dimensional Polarized Proximal Tubular Organoids with Continuous Perfusion in a Microphysiological Platform Provides Dynamic Toxicological Information in Real-Time.

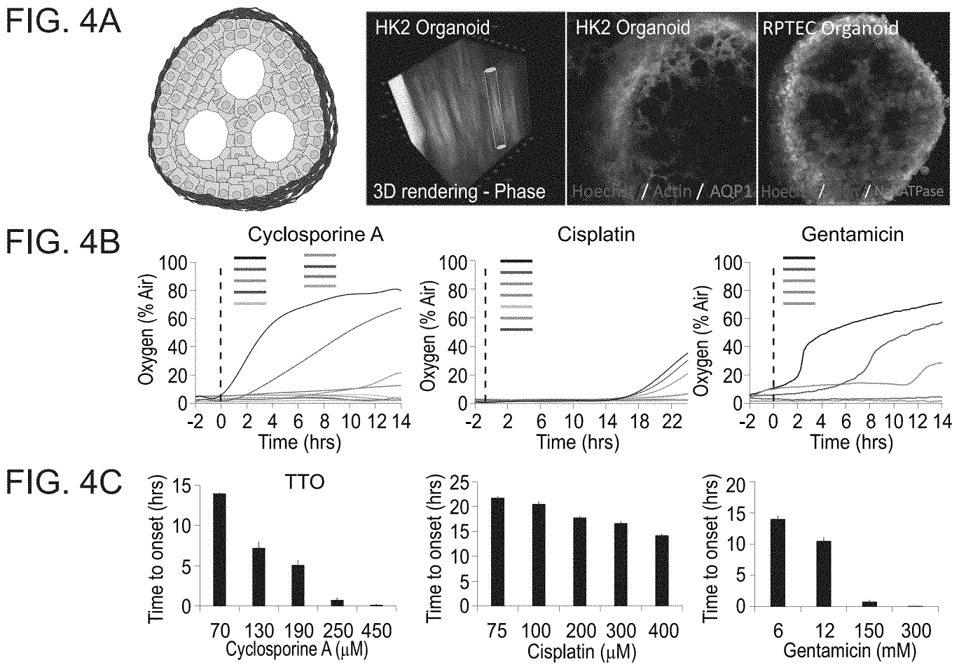

HK-2 and RPTEC vascularized organoids cultured over 4 days in an extracellular matrix form structurally mature multiple tubular structures, mimicking cortical cross-section of the human kidney. Capillaries circle around the periphery of the organoid and penetrate the tissue for more effective perfusion. Proximal tubule cells form longitudinal tubules in a unidirectional manner, as shown in a 3D reconstruction of confocal phase images of the organoid. RPTEC organoids are positive for villin, showing mature apical brush borders of polarized epithelial cells. These tissues gather critical physiological features from nephrons' S1-S2 segments of the proximal tubule, where the major part of reabsorption and excretion happens (FIG. 4A). When these organoids are embedded with microsensors for oxygen measurements and placed in the closed bioreactors under continuous perfusion, changes in their metabolism can be followed in real-time. Three different nephrotoxic FDA-approved drugs which are widely used in the clinic were perfused in the system. CsA showed acute toxicity with oxygen rising minutes after induction whereas cisplatin induced damage at least 14 hours following exposure which suggests that the compound accumulates in the cells before toxicity manifests. Gentamicin on the other hand, causes the cells to have an acute jump in oxygen uptake within an hour after exposure followed by a linear increase of oxygen uptake (FIG. 4B). Ordinate cross-sections of these data plot time-to-onsets (TTO) values. These predictive analytical values allow forecasting of tissue damage as a function of drug concentration. In this way, the length of time that results in the first signs of tissue damage can be estimated. These parameters indicate the safety margin for therapeutic use of these drugs (FIG. 4C).

Cyclosporine A, Cisplatin and Gentamicin Shift Proximal Tubule Cells Metabolism Towards Lipogenesis Upon Exposure

Cyclosporine A, Cisplatin and Gentamicin belong to different classes of drugs with different mechanisms of action. Yet, induction of subtoxic concentrations (FIG. 4B) of each of these drugs on proximal tubule cells, results in metabolic shift towards lipogenesis. Cells were exposed to each of the drugs, under constant perfusion for over 30 hours with drug concentrations provoking minimal damage (FIG. 4B). The bioreactor outflow was fluidically linked to electrochemical biosensors providing continuous measurement of glucose, lactate and glutamine levels in the medium (FIGS. 3C-G). The kinetic measurement of the flux of each metabolite is coupled in real-time with measurements of oxygen indicating how the organoids metabolism behaves upon drug exposure (FIGS. 5A-E, 6A-E and 7A-E). When exposed to CsA, the organoids' glucose uptake and lactate release are constant for the first 20 hours, suggesting that organoids use glycolysis as their ATP source. After 20 hours of exposure, lactate release decreases with a lactate over glucose ratio decreasing from 1 to 0.63, suggesting that cells shuttle their glucose towards lipid storage. Indeed, after 31 hours CsA exposure, lipogenesis had a 37.4% increase while glycolysis had a 37.1% decrease. Overall glutamine uptake stayed constant, supporting this claim (FIGS. 5A-E). Cisplatin and gentamicin showed even more drastic effects. Cisplatin caused lactate/glucose ratio to change from 0.82 to 0.26 after 31 hours of exposure. Glycolysis was decreased by 98.4% and lipogenesis was increased by 57.6%. Overall glutamine uptake remained constant (FIGS. 6A-E). Gentamicin induced changes in metabolism immediately upon exposure, resulting in a massive glucose uptake increase (+196%) and a lactate release decrease (−65.2%) after 31 hours. Glutamine uptake increased at approximately 16 hours (+76%) and returned to baseline at 31 hours (+14.1%), indicating that glutaminolysis was not a significant metabolic pathway in the response to gentamicin (FIGS. 7A-E). These three different drugs from different classes and having different mechanisms of actions converge proximal tubule cells to one common metabolic pathway for energy sustainability under exposure to toxic insult. These results suggest that proximal tubule's mode of toxicity, in response to exposure to Cyclosporine A, Cisplatin or Gentamicin perfusion, is abnormal accumulation of fat (steatosis). Fat accumulation and lipogenesis can lead to lipotoxicity which explains why these drugs are very toxic to kidneys. Furthermore, these results suggest that the proximal tubule cells compensate for stress by building lipid storage, resulting in renal steatosis.

Loss of Polarity Induces Glucose Accumulation in Proximal Tubule Cells, Glucose Transport Inhibitors Alleviate Lipotoxicity in Renal Tissue after Nephrotoxic Drug Exposure

2-NBDG is a fluorescent tracer used for monitoring glucose uptake into living cells, it consists of a glucosamine molecule substituted with a 7-nitrobenzofurazan fluorophore at its amine group. It is widely referred to a fluorescent derivative of glucose, and it is used in cell biology to visualize uptake of glucose by cells. Cells that have taken up the compound display green fluorescence. When proximal tubule cells are exposed to CsA, cisplatin and gentamicin at concentrations of a thousand-fold under the threshold of cellular damage (FIG. 4B), 2-NBDG fluorescence doubles (FIG. 8A-B). These results indicate that these cells have a detrimental glucose uptake. In mammalian cells, transport of 2-NBDG has a lower Vₘₐₓ (maximum rate), and thus the rate of transport is generally slower than glucose. This indicates that glucose accumulation is even more prominent than the changes witnessed. The witnessed loss of polarity (FIG. 2E-F) and the disturbance to normal glucose transport (FIG. 8A-B), suggest that glucose transporters such as GLUT2 cannot perform their function properly. The excess glucose is subsequently redirected towards lipid storage. Gene expression analysis indicates that after 48 hours of drug exposure, the predominant genes involved in lipogenesis such as fatty acid synthase (FASN), sterol regulatory element-binding protein 1c (SREBP1c) involved in sterol biosynthesis and HMG-CoA Reductase (HMGCR) that codes for the rate-controlling enzyme of the mevalonate pathway that produces cholesterol, are upregulated (FIG. 8C). Proximal tubule cells upregulate also their gene expression involved in β-oxydation as a coping mechanism to limit toxicity induced by the accumulation of lipids (FIG. 8C). This lipid accumulation is confirmed with lipid accumulation assay LIPIDTOX. After 48 h exposure of the three nephrotoxic drugs on both HK-2 and RPTECs, proximal tubule cells show a massive lipid accumulation with phospholipidosis in cells treated with CsA and mainly neutral lipids for cisplatin and gentamicin (FIGS. 8D-E). This strongly suggests that, like the liver, kidneys become fatty as a result of a cascade of events that converge together towards this phenomenon: loss of polarity (FIG. 2E-F), early onset of cellular injury causing mitochondrial stress (FIG. 4B-C) perturbing central carbon metabolism (FIGS. 5A-7E), pushes the cells to uptake more glucose to maintain a sustainable source of energy. The accumulation of glucose is directed towards lipid storage with upregulated gene expression of lipid biosynthesis causing downstream lipotoxicity in the kidney (FIGS. 8A-E). The present inventors hypothesized that if the increase of glucose content in the cells is the fuel for lipid accumulation in proximal tubule cells under exposure of the nephrotoxic drugs, then limiting the glucose uptake rate should alleviate the toxicity experienced by the cells and decrease overall cell death.

In proximal tubule cells, the three major glucose transporters are: GLUT2 localized on the basolateral membrane that shuttles to the apical membrane when glucose concentrations are high in the lumen of the tubule, facilitating reabsorption; SGTL1 and SGLT2 localized on the apical membrane are cotransporting glucose with sodium ions from the lumen into the cells for reabsorption of glucose into the blood. Phloretin, is a flavonoid found in extracts of apple trees leafs, it blocks the shuttling of the GLUT2 preventing its apicobasal transport. Phlorizin is also a flavonoid which is a competitive inhibitor of SGLT1 and SGLT2 know to reduce renal glucose transport and lowering glucose amount in the blood. Empagliflozin is a potent inhibitor of SGLT2 used mainly to treat type 2 diabetes, SGLT2 accounts for about 90% of glucose reabsorption into the blood. Cells were pretreated with the inhibitor alone or a mixture of all three (cocktail) for one hour before treating the cells with the drugs and the inhibitors together. CsA treated cultures, showed 2-fold increase in cell viability and a 2-fold decrease in phospholipids content in presence of phlorizin or the cocktail compared to cells treated with CsA alone. Cisplatin treated cultures, showed 20% increase in cell viability with cocktail treated cells, 20% decrease in neutral lipid content with the cocktail and 50% in phospholipid content with each inhibitor compared to cells treated with cisplatin alone. Gentamicin treated cultures, showed 65% increase in cell viability, 30% decrease in neutral lipid content and 50% in phospholipid content in the presence of the cocktail of inhibitors compared to cells treated with gentamicin alone. Cells treated with the inhibitors alone with no drugs, did not increase lipid content or induced cell death (FIG. 9A-B).

### Example 2

### Clinical Evidence Validates Kidney-On-Chip Mechanism of Drug-Induced Nephrotoxicity

As shown in Example 1, Cyclosporine A, Cisplatin and gentamicin promote early acute injury in hPTC, leading to a rapid loss of functional polarity (even below concentrations under the threshold of cellular damage). Under such conditions, polarized proximal tubules uptake more glucose to sustain glycolysis, but are unable to transport it out and therefore cannot maintain proper glucose homeostasis. Over the first 48 hours of induction, proximal tubule cells shift their metabolism towards lipogenesis leading to significant fat accumulation and lipotoxicity similar to hepatic steatosis.

Histology of human kidneys from patients under treatment with either cyclosporine A or Cisplatin show evidence of vacuolopathy in the proximal tubules which are reminiscent of lipid droplets (FIG. 10F). This clinical signs of drug-induced lipotoxicity in the proximal tubules in human patients add relevance to the present physiologically accurate kidney-on-chip platform.

A retrospective clinical study was carried out to collect data from blood and urine from patients taking either Cyclosporine A or Cisplatin alone or in combination with a SGLT2 inhibitor. Patients with hepatic or renal diseases were excluded. The average age of the patients was 48 years-old for a cohort of 247 patients (113 males and 134 females) (FIG. 10G). Lactate Dehydrogenase (LDH), a marker for cellular damage was significantly decreased to physiological levels in both males and females taking Cyclosporine A or Cisplatin in combination with the SGLT2 inhibitor (FIG. 10M-O). The same phenomenon was observed for serum creatinine (FIG. 10H-J), uric acid (FIG. 10I-K) and serum calcium (FIG. 10L-N). All of these markers significantly decreased to normal levels, suggesting that SGLT2 inhibitors alleviate drug-induced nephrotoxicity. A t-test was performed to assess statistical differences between the groups, significance was considered when p-values were less than 0.1%.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method for reducing renal tissue toxicity in a subject caused by a kidney damaging agent, the method comprising administering to the subject an agent that causes a decrease in lipid accumulation in renal tissue of the subject, thereby reducing renal toxicity caused by a kidney damaging agent in the subject, with the proviso that when the kidney damaging agent is glufosfamide, said agent that causes a decrease in lipid accumulation is not an SGLT2 inhibitor.

2. A method for reducing renal tissue toxicity in a subject caused by a kidney damaging agent, the method comprising administering to the subject an inhibitor of glucose reabsorption, thereby reducing renal toxicity caused by a kidney damaging agent in the subject, with the proviso that when the kidney damaging agent is glufosfamide, said inhibitor is not an SGLT2 inhibitor.

3. A composition comprising:
(i) a kidney-damaging agent; and
(ii) an agent that causes a decrease in lipid accumulation in renal tissue.

4. A composition comprising:
(i) a kidney-damaging agent; and
(ii) an inhibitor of glucose reabsorption of proximal tubule epithelial cells from the toxic effect of said kidney damaging agent.

5. A pharmaceutical composition comprising a pharmaceutically acceptable carrier and, as active agents the composition of claim 3.

6. A pharmaceutical composition comprising a pharmaceutically acceptable carrier and, as active agents the composition of claim 4.

7. The pharmaceutical composition of claim 5, for use in treating a disease for which the kidney damaging therapeutic agent is therapeutic.

8. The method of claim 1, wherein the subject has cancer and the kidney damaging agent is a therapeutic agent used to treat the cancer.

9. The method of claim 1, wherein the subject has undergone an organ or tissue transplant and the kidney damaging agent is an immunosuppressive agent.

10. The method of claim 1, wherein the subject has an infection and the kidney damaging agent is used to treat the infection.

11. The method of claim 1, wherein said kidney damaging agent is a therapeutic agent.

12. The method of claim 1, wherein said kidney damaging agent is a diagnostic agent.

13. The method of claim 1, wherein the subject does not have a metabolic disease.

14. The method of claim 1, wherein the subject does not have diabetes.

15. The method of claim 1, wherein the agent that causes a decrease in lipid accumulation in renal tissue is selected from the group consisting of an inhibitor of glucose reabsorption, a blocker of lipid synthesis and an up-regulator of lipid oxidation.

16. The method of claim 2, wherein said inhibitor of glucose reabsorption is selected from the group consisting of an inhibitor of Sodium-Glucose cotransporter 1 (SGLT1), an inhibitor of a sodium-glucose cotransporters 2 (SGLT2) and an inhibitor of GLUT2.

17. The method of claim 2, wherein said inhibitor of glucose reabsorption is selected from the group consisting of Phloretin, Phlorizin and empagliflozin.

18. The method of claim 1, wherein the kidney damaging agent is selected from the group consisting of an NSAID, an ACE Inhibitor, an angiotensin II Receptor Blocker, an aminoglycoside antibiotic, a radiocontrast dye, cyclosporine A (CsA) and a chemotherapeutic agent.

19. The method of claim 1, wherein the kidney damaging agent is selected from the group consisting of cisplatin, gentamicin and Cyclosporine A.

20. The method of claim 1, wherein the kidney damaging agent is a contrast agent.
